(19) United States
(12) Patent Application Publication
(10) Pub. No.: US 2019/0078162 A1
(43) Pub. Date: Mar. 14, 2019

(54) IN VITRO METHODS FOR SKIN THERAPEUTIC COMPOUND DISCOVERY USING SKIN AGE BIOMARKERS

(71) Applicant: OneSkin Technologies, Inc., San Francisco, CA (US)

(72) Inventors: Carolina Reis de Oliveira, San Francisco, CA (US); Edgar Andres Ochoa Cruz, San Francisco, CA (US); Juliana Lott de Carvalho, Lago Norte (BR); Alessandra Arcoverde Cavalcanti Zonari, Coimbra (PT); Mariana Lima Boroni Martins, Rio de Janeiro (BR); Daniel Austin Foyt, San Francisco, CA (US)

(21) Appl. No.: 16/132,297

(22) Filed: Sep. 14, 2018

Related U.S. Application Data
(60) Provisional application No. 62/558,734, filed on Sep. 14, 2017.
(60) Provisional application No. 62/577,106, filed on Oct. 25, 2017.

(51) Int. Cl. C12Q 1/6883; G01N 33/50

(52) U.S. Cl. CPC C12Q 1/6883; G01N 33/5088; C12Q 2600/154; C12Q 2600/158; C12Q 2600/148

(57) ABSTRACT

In one aspect, a method for identifying age-preventing agents for skin is disclosed. Skin samples are transferred to a first vessel, a second vessel, and a third vessel. A skin age inducing agent is applied to the skin samples in the first vessel and the second vessel. A prospective age-preventing agent is applied to the skin sample in the first vessel. Genetic material is extracted from the skin samples in the first vessel, the second vessel and the third vessel. A quantity of a skin age biomarker is measured in the extracted genetic material from each vessel. A score is determined for the skin samples in the first vessel, the second vessel and the third vessel based on the quantity of skin age biomarker measured in the genetic material extracted from the skin sample in each vessel, wherein the score is indicative of a condition of the skin sample in each vessel.

### APPLICATIONS FOR CLAIM OF PRIORITY

This application claims the benefit under 35 U.S.C. § 119(e) of U.S. Provisional Application Ser. No. 62/558,734 filed September 14, 2017 and U.S. Provisional Application Ser. No. 62/577,106 filed Oct. 25, 2017. The disclosures of the above-identified applications are incorporated herein by reference as if set forth in full.

### FIELD

The embodiments disclosed herein are generally directed towards in vitro methods for discovering therapeutic compounds for tissue (e.g., skin, brain, liver, eye, cardiac, cancerous or diseased, etc.) using tissue age biomarkers (e.g., epigenetic markers, gene expression markers, etc). More specifically, there is a need for in vitro methods for screening compounds for efficacy in preventing or reversing (i.e., rejuvenating) skin aging using objective measures (e.g., epigenetic markers, gene expression analysis, etc.) that can quantify skin age.

### BACKGROUND

Aging and longevity are influenced by a myriad of processes. In fact, unraveling the molecular mechanisms of aging remains one of the fundamental and most complex problems in biology. Considering that there is no way of preventing time to pass, it is important to differentiate healthy aging from early onset or pathological aging, as well as to quantify aging in order to characterize normal and abnormal events. Adding complexity to this scenario, not only do environmental factors (exposome) heavily influence aging, but also genetic factors, rendering the measurement of aging a rather challenging task.

Conventional methods to measure skin aging are primarily image-based techniques that are designed to visually detect levels of skin imperfections and wrinkles (e.g., KR1020110011009, JP2010115131, etc.) to predict skin age. Additionally, conventional methods have been developed to correlate epigenetic alterations (that occur throughout an individual's life) with aging for use as a kind of “molecular clock” to measure age of an individual as described in U.S. Patent Application Publication Number US20140228231 entitled “Method to Estimate Age of Individual Based on Epigenetic Markers in Biological Sample.” The methods call for analyzing the methylation status of 88 loci from the DNA of epithelial or white blood cells isolated from a patient to estimate the age of the patient. Interestingly, in the associated publication (Horvath Genome Biology 2013, 14:R115), it was stated that there is a “poor correlation” between DNA methylation of a donor's dermal fibroblasts and his/her age.

More recently, studies involving methods that analyze up to 450,000 methylation markers have been shown to provide relatively good predictions of female skin age, but these methods are invasive (require biopsying the test subject's skin), labor intensive and highly expensive; which hinders its application for screening promising compounds for their therapeutic effects on skin age (Bormann F et al., Aging Cell (2016) 15, pp563-571).

As such, there is a need for in vitro based techniques to screen compound candidates for efficacy in preventing or reversing (i.e., rejuvenation) tissue (i.e., skin, brain, liver, eye, cardiac, cancerous or diseased, etc.) aging (therapeutic effects on tissue age) using objective measures (e.g., epigenetic markers, gene expression analysis, etc.) that can quantify tissue age.

### SUMMARY

In one aspect, a method for identifying age-preventing agents for skin is disclosed. Skin samples are transferred to a first vessel, a second vessel, and a third vessel. A skin age inducing agent is applied to the skin samples in the first vessel and the second vessel. A prospective age-preventing agent is applied to the skin sample in the first vessel. Genetic material is extracted from the skin samples in the first vessel, the second vessel and the third vessel. A quantity of a skin age biomarker is measured in the extracted genetic material from each vessel. A score is determined for the skin samples in the first vessel, the second vessel and the third vessel based on the quantity of skin age biomarker measured in the genetic material extracted from the skin sample in each vessel, wherein the score is indicative of a condition of the skin sample in each vessel.

In another aspect, a method for identifying age-reversing agents for skin is disclosed. Aged skin samples are transferred to a first vessel and a second vessel. A prospective age-reversing agent is applied to the aged skin sample in the first vessel. Genetic material is extracted from the aged skin samples in the first vessel and the second vessel. A quantity of a skin age biomarker is measured in the extracted genetic material from each vessel. A score is determined for the aged skin samples in the first vessel and the second vessel based on the quantity of the skin age biomarker measured in the genetic material extracted from the aged skin sample in each vessel, wherein the score is indicative of a condition of the aged skin sample in each vessel.

In yet another aspect, a method for predicting skin age is disclosed. Genetic material is extracted from a skin sample. A quantity of a skin age biomarker is measured in the extracted genetic material. A score is determined for the skin sample based on the quantity of the skin age biomarker measured in the extracted genetic material, wherein the score is indicative of a condition of the skin sample.

In yet another aspect, a method for identifying age-preventing agents for biological tissue is disclosed. Tissue samples are transferred to a first vessel, a second vessel, and a third vessel. A tissue age inducing agent is applied to the tissue samples in the first vessel and the second vessel. A prospective age-preventing agent is applied to the tissue sample in the first vessel. Genetic material is extracted from the tissue samples in the first vessel, the second vessel and the third vessel. A quantity of a tissue age biomarker is measured in the extracted genetic material from each vessel. A score is determined for the tissue samples in the first vessel, the second vessel and the third vessel based on the quantity of tissue age biomarker measured in the genetic material extracted from the tissue sample in each vessel, wherein the score is indicative of a condition of the tissue sample in each vessel.

In yet another aspect, a method for identifying age-reversing agents for biological tissue is disclosed. Aged tissue samples are transferred to a first vessel and a second vessel. A prospective age-reversing agent is applied to the aged tissue sample in the first vessel. Genetic material is extracted from the aged tissue samples in the first vessel and the second vessel. A quantity of a tissue age biomarker is measured in the extracted genetic material from each vessel. A score is determined for the aged tissue samples in the first vessel and the second vessel based on the quantity of the tissue age biomarker measured in the genetic material extracted from the aged tissue sample in each vessel, wherein the score is indicative of a condition of the aged tissue sample in each vessel.

In yet another aspect, a method for predicting tissue age is disclosed. Genetic material is extracted from a tissue sample. A quantity of a tissue age biomarker is measured in the extracted genetic material. A score is determined for the tissue sample based on the quantity of the tissue age biomarker measured in the extracted genetic material, wherein the score is indicative of a condition of the tissue sample.

Preferably, the skin tissue comprises dermis and/or epidermis.

Preferably, the skin age biomarker comprises genes or gene expression products comprising mRNA or protein. Especially, the skin age biomarker includes changes in expression of one or more of the genes in Tables 1-3; e.g., at least 1, 2, 3, 4, 5, 6, 7 or 8 of the genes of Tables 1-3.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 illustrates a workflow for screening and discovery of skin age-preventing compounds, in accordance with some embodiments of the disclosure.

FIG. 2 illustrates a workflow for screening and discovery of skin age-reversing compounds, in accordance with some embodiments of the disclosure.

FIG. 3 is a block diagram that illustrates a computer system, in accordance with various embodiments.

FIG. 4 is a flowchart showing a method for identifying age-preventing agents for skin, in accordance with various embodiments.

FIG. 5 is a flowchart showing a method for identifying age-reversing agents for skin, in accordance with various embodiments.

FIG. 6 is a flowchart showing a method for predicting skin age, in accordance with various embodiments.

FIG. 7 is photomicrograph showing cross-section skin tissue of neonate, a 29 year-old subject and an 84 year-old subject.

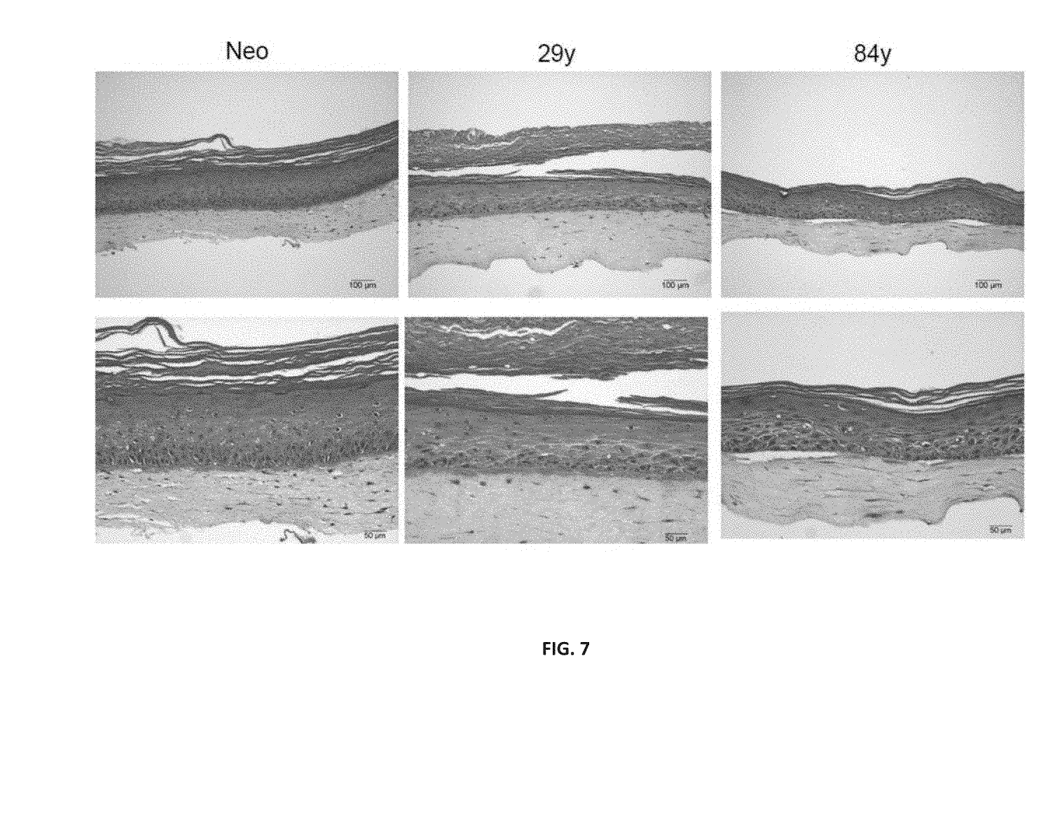

FIG. 8 shows relative expression of various genes in the dermis. *p<0.05, **p<0.01, ***p<0.001,**** p<0.0001 one-way ANOVA, Dunnett's multiple comparison test.

FIG. 9 shows relative expression of various genes in the epidermis. *p<0.05, **p<0.01, ***p<0.001,**** p<0.0001 one-way ANOVA, Dunnett's multiple comparison test.

FIG. 10 shows a schematic diagram of the methodology used in testing for age reversal compounds.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As used herein, the term “detecting,” refers to the process of determining a value or set of values associated with a sample by measurement of one or more parameters in a sample, and may further comprise comparing a test sample against reference sample. In accordance with the present disclosure, the detection of tumors includes identification, assaying, measuring and/or quantifying one or more markers.

As used herein, the term “subject” means an individual. In one aspect, a subject is a mammal such as a human. In one aspect a subject can be a non-human primate. Non-human primates include marmosets, monkeys, chimpanzees, gorillas, orangutans, and gibbons, to name a few. The term “subject” also includes domesticated animals, such as cats, dogs, etc., livestock (e.g., cows, pigs, goats), laboratory animals (e.g., mouse, rabbit, rat, gerbil, guinea pig, etc.) and avian species (e.g., chickens, turkeys, ducks, etc.). Subjects can also include, but are not limited to fish (for example, zebrafish, goldfish, tilapia, salmon, and trout), amphibians and reptiles. Preferably, the subject is a human subject. Especially, the subject is a human patient.

The term “age-associated disorder” in the context of a “subject” is used to describe a disorder observed with the biological progression of events occurring over time in a subject. Preferably, the subject is a human. Non-limiting examples of age-associated disorders include, but are not limited to, hypertension, atherosclerosis, diabetes mellitus, dementia, skin disorders or structural alterations. An age-associated disorder may also be a cell proliferative disorder. Examples of age-associated disorders which are cell proliferative disorders include colon cancer, lung cancer, breast cancer, prostate cancer, and melanoma, amongst others. An age-associated disorder is further intended to mean the biological progression of events that occur during a disease process that affects the body, which mimic or substantially mimic all or part of the aging events which occur in a normal subject, but which occur in the diseased state over a shorter period of time. Particularly, the age-associated disorder is a “memory disorder” or “learning disorder” which is characterized by a statistically significant decrease in memory or learning assessed over time. In some embodiments, the age-associated disorder is a skin disorder, e.g., wrinkles, lines, dryness, itchiness, age-spots, bedsores, dyspigmentation, infection (e.g., fungal infection), and/or a reduction in a skin property selected from clarity, texture, elasticity, color, tone, pliability, firmness, tightness, smoothness, thickness, radiance, evenness, laxity, and oiliness.

The term “sample” as used herein refers to a composition that is obtained or derived from a subject of interest that contains a cellular and/or other molecular entity that is to be characterized and/or identified, for example based on physical, biochemical, chemical and/or physiological characteristics. Preferably, the sample is a “biological sample,” which means a sample that is derived from a living entity, e.g., cells, tissues, organs, in vitro engineered organs and the like. In some embodiments, the source of the tissue sample may be blood or any blood constituents; bodily fluids; solid tissue as from a fresh, frozen and/or preserved organ or tissue sample or biopsy or aspirate; and cells from any time in gestation or development of the subject or plasma. Samples include, but not limited to, primary or 2D and 3D cultured cells or cell lines, cell supernatants, cell lysates, platelets, serum, plasma, vitreous fluid, ocular fluid, lymph fluid, synovial fluid, follicular fluid, seminal fluid, amniotic fluid, milk, whole blood, urine, cerebrospinal fluid (CSF), saliva, sputum, tears, perspiration, mucus, tumor lysates, skin punch or biopsy, and tissue culture medium, as well as tissue extracts such as homogenized tissue, tumor tissue, and cellular extracts. Samples further include biological samples that have been manipulated in any way after their procurement, such as by treatment with reagents, solubilized, or enriched for certain components, such as proteins or nucleic acids, or embedded in a semi-solid or solid matrix for sectioning purposes, e.g., a thin slice of tissue or cells in a histological sample. Samples may contain environmental components, such as, e.g., water, soil, mud, air, resins, minerals, etc. In certain embodiments, a sample may comprise biological specimen containing DNA (for example, genomic DNA or gDNA), RNA (including mRNA, tRNA and all other classes), protein, or combinations thereof, obtained from a subject (such as a human or other mammalian subject).

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

As used herein, the term “locus” refers to a specific position along a chromosome or DNA sequence. Depending upon context, a locus could be a gene, a marker, a chromosomal band or a specific sequence of one or more nucleotides. Typically, loci are in proximity to the genes/markers they are associated with, e.g., within 5 kilo bases (kb), within 4 kb, within 2 kb, within 1 kb, within 800 base pairs (bp), within 500 bp, within 400 bp, within 300 bp, within 200 bp, within 100 bp, within 50 bp, within 30 bp, within 20 bp, or fewer bp of named gene or CpG.

As used herein, the term “allele” refers to one of a pair or series, of forms of a gene or non-genic region that occur at a given locus in a chromosome. In a normal diploid cell there are two alleles of any one gene (one from each parent), which occupy the same relative position (locus) on homologous chromosomes. Within a population there may be more than two alleles of a gene. SNPs also have alleles, e.g., the two (or more) nucleotides that characterize the SNP.

As used herein, the term “probe” refers to a nucleic acid or oligonucleotide that forms a hybrid structure with a sequence in a target region of a nucleic acid due to complementarity of the probe or primer sequence to at least one portion of the target region sequence.

The term “label” as used herein refers, for example, to a compound that is detectable, either directly or indirectly. The term includes colorimetric (e.g., luminescent) labels, light scattering labels or radioactive labels. Fluorescent labels include, inter alia, the commercially available fluorescein phosphoramidites such as FLUOREPRIME™ (Pharmacia™) FLUOREDITE™ (Millipore™) and FAM™ (ABI™) (see, e.g., U.S. Pat. No. 6,287,778).

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer may range from, e.g., 10 to 50 nucleotides; preferably 12 to 30 nucleotides.

The term “complementary” as used herein refers to the hybridization or base pairing, e.g., via hydrogen bonds, between nucleotides or nucleic acids, such as, for instance, between the two strands of a double stranded DNA molecule or between an oligonucleotide primer and a primer. Complementary polynucleotides may be aligned at least 70%, at least 80%, at least 90%, at least 95%, at least 98%, at least 99% or a greater percentage, e.g., 99.9%.

The term “hybridization,” as used herein, refers to any process by which a strand of nucleic acid bonds with a complementary strand through base pairing. For example, hybridization under high stringency conditions could occur in about 50% formamide at about 37° C. to about 42° C. Hybridization could occur under reduced stringency conditions in about 35% to 25% formamide at about 30° C. to 35° C. In particular, hybridization could occur under high stringency conditions at 42° C. in 50% formamide, 5xSSPE, 0.3% SDS, and 200 μg/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature. Variations on the above ranges and conditions are well known in the art.

The term “hybridization complex” as used herein, refers to a complex formed between two nucleic acid sequences by virtue of the formation of hydrogen bonds between complementary bases. A hybridization complex may be formed in solution or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

As used herein, the term “marker” refers to a characteristic that can be objectively measured as an indicator of normal biological processes, pathogenic processes or a pharmacological response to a therapeutic intervention, e.g., treatment with an anti-cancer agent. Representative types of markers include, for example, molecular changes in the structure (e.g., sequence) or number of the marker, comprising, e.g., gene mutations, gene duplications, or a plurality of differences, such as somatic alterations in gDNA, copy number variations, tandem repeats, gene expression level or a combination thereof. The term “marker” includes products of genes, e.g., mRNA transcript and the protein product, including variants thereof, such as, for example, splice variants of primary mRNA and the polypeptide products thereof. Markers include differentially expressed gene products, e.g., over-expression, under-expression, knockout, constitutive expression, mistimed expression, compared to controls. Markers of the disclosure further include cis-regulatory elements and/or trans-regulatory elements. As is known in the art, “cis-regulatory elements” are present on the same molecule of DNA as the gene they regulate whereas “trans-regulatory elements” can regulate genes distant from the gene from which they were transcribed. Representative examples of cis-regulatory elements include, e.g., promoters, enhancers, repressors, etc. Representative examples of trans-regulatory elements include e.g., DNA sequences that encode transcription factors. The trans-regulation or cis-regulation could be at the level of transcription or methylation. In some embodiments, cis-regulatory elements are often binding sites for one or more trans-acting factors.

As used herein, the term “methylation” will be understood to mean the presence of a methyl group added to a nucleotide. The nucleobases of DNA/RNA can be derivatized. DNA methylation refers to the addition of a methyl (CH₃) group to the DNA strand itself, often to the fifth carbon atom of a cytosine ring.

The term “methylation marker” as used herein refers to a CpG position that is potentially methylated. Methylation typically occurs in a CpG containing nucleic acid. The CpG containing nucleic acid may be present in, e.g., in a CpG island, a CpG doublet, a promoter, an intron, or an exon of gene. For instance, in the genetic regions provided herein the potential methylation sites encompass the promoter/enhancer regions of the indicated genes. Thus, the regions can begin upstream of a gene promoter and extend downstream into the transcribed region.

The term “methylation status” as used herein refers to the presence or absence of methylation in a specific nucleic acid region e.g., genomic region. In the context of the present disclosure, the term “methylation status” encompasses methylation status or hydroxymethylation status of “—C—phosphate-G-” (CpG) sites or “—C-phosphate-any base (N)-phosphate-G” (CpNpG) sites and genes. The term “methylation status” also encompasses methylation status of non CpG sites or non-CG methylation. In particular, the present disclosure relates to detection of “methylation status” of cytosine (5-methylcytosine). A nucleic acid sequence may comprise one or more such CpG methylation sites.

In some embodiments, the “methylation status” is indicative of a level of the methylation in a nucleic acid. Herein, the methylation level may be expressed in any numeric form, e.g., total count, arithmetic mean, e.g., average per million base pairs (bp), geometric mean, etc. Counts may be obtained using, e.g., quantitative bisulfite pyrosequencing with the PSQ HS 96A pyrosequencing system (Qiagen, Germantown, Md., USA) following bisulfite modification of genomic DNA using EZ DNA methylation GOLD KITS (Zymo Research, Irvine, Calif., USA).

The term “gene expression profile” refers to a representation of the expression level of a plurality of genes in response to a selected expression condition (for example, incubation in the presence of a standard compound or test compound). Gene expression profiles can be expressed in terms of an absolute quantity of mRNA transcribed for each gene, as a ratio of mRNA transcribed in a test cell as compared with a control cell, and the like. As used herein, a “standard” gene expression profile refers to a profile already present in the primary database (for example, a profile obtained by incubation of a test cell with a standard compound, such as a drug of known activity), while a “test” gene expression profile refers to a profile generated under the conditions being investigated. The term “modulated” refers to an alteration in the expression level (induction or repression) to a measurable or detectable degree, as compared to a pre-established standard (for example, the expression level of a selected tissue or cell type at a selected phase under selected conditions).

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ and NEXTSEQ Systems of Illumina and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

As used herein the term “whole transcriptome sequencing” refers to determining the expression of all RNA molecules including messenger RNA (mRNA), ribosomal RNA (rRNA), transfer RNA (tRNA), and non-coding RNA. Whole transcriptome sequencing can be done with a variety of platforms for example, the Genome Analyzer (Illumina, Inc., San Diego, Calif., USA) and the SOLID™ Sequencing System (Life Technologies, Carlsbad, Calif., USA). However, any platform useful for whole transcriptome sequencing may be used.

The term “RNA-Seq” or “transcriptome sequencing” refers to sequencing performed on RNA (or cDNA) instead of DNA, where typically, the primary goal is to measure expression levels, detect fusion transcripts, alternative splicing, and other genomic alterations that can be better assessed from RNA. RNA-Seq includes whole transcriptome sequencing as well as target specific sequencing.

Genomic variants can be identified using a variety of techniques, including, but not limited to: array-based methods (e.g., DNA microarrays, etc.), real-time/digital/quantitative PCR instrument methods and whole or targeted nucleic acid sequencing systems (e.g., NGS systems, Capillary Electrophoresis systems, etc.). With nucleic acid sequencing, coverage data can be available at single base resolution.

Various aspects and embodiments of the methods and systems disclosed herein use conventional and specialized sequence alignment methods that can align a fragment sequence to a reference sequence or another fragment sequence. The fragment sequence can be obtained from a fragment library, a paired-end library, a mate-pair library, a concatenated fragment library, or another type of library that may be reflected or represented by nucleic acid sequence information including for example, RNA, DNA, and protein based sequence information. Generally, the length of the fragment sequence can be substantially less than the length of the reference sequence. The fragment sequence and the reference sequence can each include a sequence of symbols. The alignment of the fragment sequence and the reference sequence can include a limited number of mismatches between the symbols of the fragment sequence and the symbols of the reference sequence. Generally, the fragment sequence can be aligned to a portion of the reference sequence in order to minimize the number of mismatches between the fragment sequence and the reference sequence.

Information related to the genetic features may be obtained using routine means. For instance, using University of California Santa Cruz's Genome Browser on Human (GRCh38/hg38) Assembly (assembled: December 2013), which is accessible on the web at genome(dot)ucsc(dot)edu/cgi-bin/hgGateway. Therein, an assembly is selected (e.g., Genome Reference Consortium Human Build 38 (GRCh38) and under the search field, the gene name or the chromosome number and the region may be specified.

The term “epigenetic” as used herein means relating to, being, or involving a modification in gene expression that is independent of DNA sequence. Epigenetic factors can include modifications in gene expression that are controlled by changes in DNA methylation and chromatin structure and/or by the presence or absence of non-coding RNAs (e.g., miRNA, sRNA, siRNA, etc.). For example, DNA methylation patterns and the quantity of non-coding RNAs in a sample are known to correlate with gene expression.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). The present disclosure contemplates any deoxyribonucleotide (DNA), ribonucleotide (RNA) or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “test compound” refers in general to a compound to which a test cell is exposed, about which one desires to collect data. Typical test compounds will be small organic molecules, typically prospective pharmaceutical lead compounds, but can include proteins, peptides, polynucleotides, heterologous genes (in expression systems), plasmids, polynucleotide analogs, peptide analogs, lipids, carbohydrates, viruses, phage, parasites, and the like.

The term “biological activity” as used herein refers to the ability of a test compound to alter the expression of one or more genes.

The term “test cell” refers to a biological system or a model of a biological system capable of reacting to the presence of a test compound, typically a eukaryotic cell or tissue sample, or a prokaryotic organism.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

In various embodiments, a computer program product can include instructions to select a contiguous portion of a fragment sequence; instructions to map the contiguous portion of the fragment sequence to a reference sequence using an approximate string matching method that produces at least one match of the contiguous portion to the reference sequence.

The term “screening”, as used herein, refers to an assay to assess the genotype or phenotype of a cell or cell product including, but not limited to nucleic acid sequence (e.g., levels of mRNA or variant thereof such as splice variants), protein sequence, protein function (e.g., binding, enzymatic activity, blocking activity, cross-blocking activity, neutralization activity, and the like). The assays include gene expression assays (e.g., hybridization assays, Northern blots, DNA microarray, sequencing, amplification) ELISA-based assays, BIACORE assays, etc.

The term “positive”, as used herein, refers to identification of a parameter (e.g., the expression of an mRNA or protein in a cell), which greater than by at least 5% (e.g., 10%, 20%, 30%, 50%, 75%, 100%, 200%, 300%, 500%, or more, e.g., 10-fold, 20-fold or 50-fold) of a control (e.g., expression of the same mRNA or protein in a control cell, e.g., untreated cell).

The term “negative”, as used herein, refers to identification of a parameter (e.g., the expression of an mRNA or protein in a cell), which less than 5% (e.g., 4%, 3%, 2%, 1%) of a control (e.g., expression of the same mRNA or protein in a control cell, e.g., untreated cell).

The term “compounds” used in screening include any small molecule or large molecule compounds. The term “small molecule compound” includes compounds that are typically smaller than 5 KDa, e.g., organic compounds, peptides, vitamins, minerals. The term “large molecule compound” includes compounds that are typically larger than 5 KDa, e.g., proteins, antibodies. Compounds include agents known to have desired biological effects, e.g., retinoic acid which is known to accelerate aging.

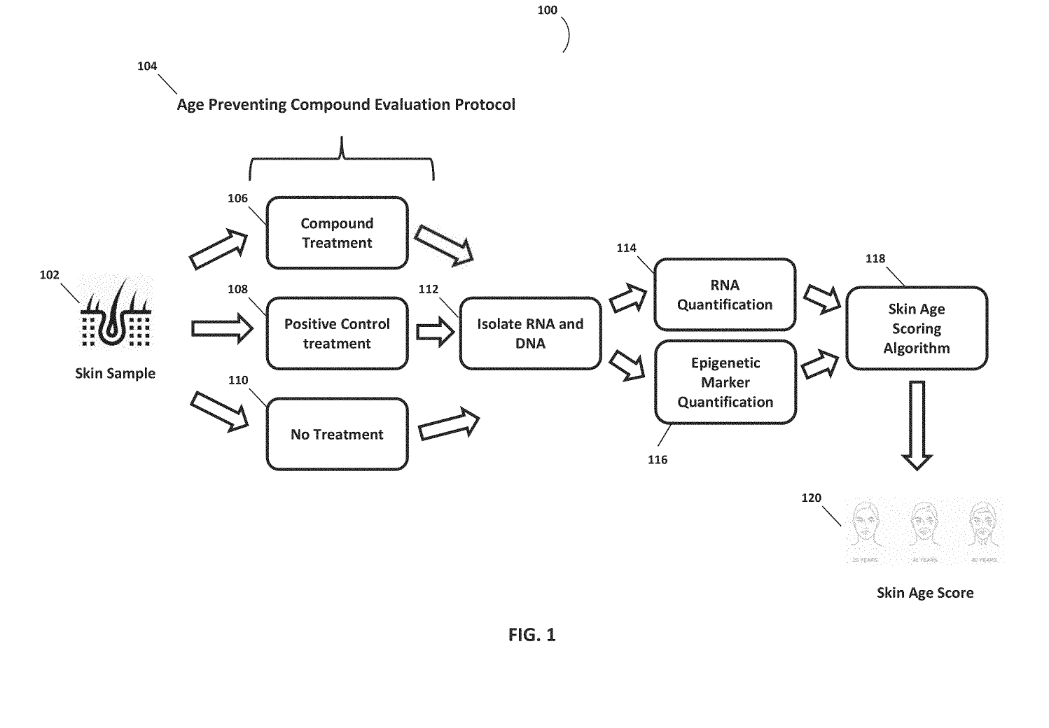

FIG. 1 illustrates a workflow 100 for screening and discovery of skin age-preventing compounds, in accordance with some embodiments of the disclosure. As depicted herein, artificially grown skin samples 102 are transferred into three separate vessels (106, 108 and 110) that are used in an age preventing compound evaluation protocol 104. The skin samples 102 can be a suspension of non-tissue skin cells, an agglomeration of skin cells or skin tissue.

In some embodiments, the artificial grown skin sample 102 originates from the epidermis layer of skin. In other embodiments, the artificially grown skin sample 102 originates from the dermis layer of skin. In still other embodiments, the artificially grown skin sample 102 originates from the hypodermis layer of skin. That is, the artificially grown skin 102 can be grown from cells or tissue grafts originating from the epidermal, dermal and/or hypodermal layers of skin.

Some examples of a sample vessel may include, but are not limited to, a test tube, pipette tube, petri dish, or a well/partition within a multi-partition/well plate.

In some embodiments, the artificial skin samples 102 originate from the same batch of artificially grown skin. That is, the skin samples 102 transferred to each of the three vessels originate from the same source of skin cells/tissue that was grown in a single batch. In other embodiments, the artificial skin samples 102 originate from two or more batches of artificially grown skin. That is, the skin samples 102 transferred to each vessel can originate from skin grown in different batches. It should be understood, however, that the skin samples 102 can be from any batch source as long as they are in substantially same physiological or cell/tissue aging condition.

After the skin samples 102 have been transferred, a skin age inducing agent (e.g., doxorubicin, ionizing radiation, H₂O₂, etc.) is applied to the skin samples 102 in the first (‘Compound Treatment’) vessel 106 and the second (‘Positive Control Treatment’) vessel 108. In addition, a prospective age-preventing agent (e.g., compound, composition, biologic, etc.) is applied to the skin sample 102 in the first vessel 106. In some embodiments, the skin age inducing agent and the prospective age-preventing agent are contemporaneously applied to the skin sample 102 in the first vessel. In other embodiments, the prospective age-preventing agent is applied to the skin sample 102 in the first vessel 106 a set period of time before or after the skin age inducing agent is applied to the skin sample 102 in the first vessel 106. In some embodiments, the set period of time is between about 24 hours to about 120 hours. In other embodiments, the set period of time is between about 36 hours to about 96 hours. In still other embodiments, the set period of time is between about 48 hours to about 72 hours. It should be understood, however, that the set period of time can essentially be as long a period of time as is required to determine the effectiveness of the prospective age-preventing agent.

In some embodiments, the prospective age-preventing agent is an agent (e.g. compound, composition, biologic, etc.) that prevents cellular aging, inhibits cell apoptosis/necrosis or reduces cell oxidative stress. In some exemplary embodiments, the agent is an activator of cell apoptosis/necrosis by inhibiting B-cell Lymphoma (bcl) activity.

After the application of the skin age inducing agent (the first 106 and second vessels 108) and the prospective age-preventing agent (the first vessel 106 only), the skin samples 102 in all three vessels (the first 106, the second 108 and the third 110) are incubated for a set period of time. In some embodiments, the vessels are incubated for between about 24 hours to about 120 hours. In other embodiments, the vessels are incubated for between about 36 hours to about 96 hours. In still other embodiments, the vessels are incubated for between about 48 hours to about 72 hours. It should be understood, however, the vessels can essentially be incubated for as long a period of time as is necessary to determine the effectiveness of the prospective age-preventing agent.

Upon the completion of the incubation period, genetic material (DNA and RNA) is individually extracted 112 from the skin samples 102 in all three vessels (106, 108, 110), processed using an appropriate sample library preparation protocol and analyzed on a NGS (or equivalent) type of genomic sequencing system, microarray (DNA or RNA), qPCR, dPCR, etc., to measure the quantity of one or more skin age biomarkers (e.g., RNA 114 and/or epigenetic marker 116) in each of the skin samples 102.

In some embodiments, the extracted genetic material is cellular genetic material. That is, genetic material that is found within the cells comprising the skin samples 102. In other embodiments, the extracted genetic material is extracellular genetic material. That is, genetic material that is either shed or secreted by the cells comprising the skin samples 102.

In some embodiments, the skin age biomarker is an epigenetic modification that is measured as a quantity of epigenetically modified DNA 116 found in the genetic material extracted from each of the skin samples 102. In various embodiments, the epigenetic modification is a covalent-type DNA modification. Examples of covalent-type DNA modifications include, but are not limited to: methylation, acetylation, ubiquitylation, phosphorylation, sumoylation, ribosylation, citrullination, etc. In various embodiments, the epigenetic modification is a histone modification.

In some embodiments, the skin age biomarker measured is a quantity of RNA 114 found in the genetic material extracted from each of the skin samples 102. In various embodiments, the RNA measured is messenger RNA (mRNA). In various embodiments, the RNA measured is a non-coding RNA. Examples of non-coding RNA include, but are not limited to: siRNA, sRNA, microRNA, tRNA, rRNA, etc.

In still other embodiments, the quantities of a combination of different skin age biomarkers are measured in the genetic material extracted from each of the skin samples 102. For example, the quantities of both DNA methylation and mRNA can be measured in the genetic material extracted from each of the skin samples 102.

Once the quantity of a skin age biomarker has been measured for a skin sample 102, the value is input into a skin age scoring algorithm 118 which determines a score 120 that correlates with a condition (e.g., physiological/structural, functional, metabolic, etc.) that the skin sample 102 is in. In some embodiments, the condition is the predicted age of the skin sample 102. In other embodiments, the condition is the level of cellular aging (e.g., cell damage) in the skin sample 102. In still other embodiments, the condition is the level of cell death (e.g., apoptosis or necrosis) in the skin sample 102. In still other embodiments, the condition is a level of proliferative cells in the skin sample 102.

In some embodiments, the prospective age-preventing agent is classified as an age-preventing agent if the score 102 determined for the skin sample 102 in the first vessel 106 is less than the score 120 determined for the skin sample 102 in the second vessel 108. In other embodiments, the prospective age-preventing agent is classified as an age-preventing agent if the score 120 determined for the skin sample 102 in the first vessel 106 is less than the score 120 determined for the skin sample 102 in the second vessel 108 and substantially similar to the score 120 determined for the skin sample 102 in the third (‘No Treatment’) vessel 110.

It should be appreciated that, with some modifications, the compound discovery workflows disclosed herein, can also be broadly used for screening and discovery of compounds that may be useful in preventing or curing (i.e., reversing) a number of well known age-related diseases and conditions.

For example:

### Macular Degeneration

Age Macular Degeneration (AMD) constitutes a leading cause of blindness in industrialized countries, affecting approximately 8% of the population within ages 45-85 years. It is estimated that 196 million affected people in 2020. AMD's primary cause is the loss of retinal pigmented cells, which leads to photoreceptor death.

It is well documented in medical literature that, with age, both photoreceptors and the retinal pigment epithelium show slow degenerative changes, followed by their demise and often accompanied by the development of a neovascular membrane. Moreover, chronic and repetitive non-lethal retinal pigment epithelium (RPE) injuries (together with an oxidative environment) appear to be important factors for development of AMD.

Cellular senescence (i.e., aging) has also been associated with the disease, which may corroborate the role of aging in this pathology. In vitro evidence supports this hypothesis, being that, the exposure of RPE cells to senescence-inducing stimuli, such as H₂O₂, promotes senescence-associated secretory phenotype (SASP) expression which is characterized by the production and release of specific soluble molecules, such as pro-inflammatory cytokines which are linked to AMD pathogenesis.

Despite this evidence, no evaluation of the age-related biomarkers (e.g., epigenetic, genetic, etc.) of the RPE cells has been performed. Also, by collecting tissue of AMD and non-AMD donors, it will be possible to confirm the hypothesis that precocious senescence may cause AMD and that anti-aging strategies may successfully prevent AMD.

Although much progress has been made recently in the management of the later stages of AMD, no agents have yet been developed for the early stages or for prophylactic use. This might be finally achieved thru prevention of cellular senescence.

### Dementia

Considering age-related cognitive decline, age is the primary risk factor for many neurodegenerative diseases including Alzheimer's disease (AD), Parkinson's disease and dementia, which is an umbrella term used to describe diseases that cause dysfunction or death of neurons. Neural cells in AD patients show strong immunoreactivity for p16Ink4a a biomarker of aging which is not presented in non-senescent, terminally differentiated neurons. Also, telomeres tend to be shorter in patients with dementia compared to healthy ones and senescent astrocytes contribute to AD. Age-related biomarkers (e.g., epigenetic, genetic, etc.) of the brain is currently a target of research, being that such molecular evidence of aging is highly associated to cognitive decline. Therefore, there is increasing evidence that cellular senescence (i.e., aging) may be related to neuron dysfunction associated to dementia.

Despite such evidence, current studies are mainly observational and do not propose interventional strategies. By measuring age-related biomarkers (e.g., epigenetic, genetic, etc.) of brain tissue prior to and after molecule testing, it may be possible to screen novel molecules with anti-aging potential for the brain, and, possibly, preventive effect over such pathology.

### Atherosclerosis

Atherosclerosis is frequently the underlying cause of cardiovascular diseases, which are the primary cause of mortality in the Western world. This disease is highly influenced by age, in addition to environmental factors. Corroborating such observation, it has been well documented in medical literature that, during atherosclerotic plaque formation and expansion, senescent (i.e., aged) vascular smooth muscle and endothelial cells can be found. Two mechanisms of senescence induction in this context are cellular proliferation, as well as oxidative stress. Because of the complex signaling between endothelial and smooth muscle cells, and immune cells recruited to plaques, these findings raise the possibility of a multistep role of senescent cells in atherogenesis and the possibility that anti-aging therapeutic compounds may be discovered to prevent or reverse atherosclerosis.

### Cancer

Cancer constitutes a pathology associated with cellular proliferation, independently from external stimuli. Most cancers are associated with aging. Confirming such an observation, DNA aging (as quantified by age-related biomarkers) has been linked with cancer risk factors (e.g., breast cancer risk) which raises the possibility that anti-aging therapeutic compounds may be discovered to prevent or cure cancer.

In view of the potential compound screening and discovery applications discussed herein, the compound discovery workflow of FIG. 1 can be modified to allow for the screening and discovery of tissue age-preventing compounds that can be used to prevent age-related diseases and conditions.

For example, any type (e.g., muscle, cartilage, fat, skin, liver, lung, neural/brain, cancerous or diseased, etc.) of artificially grown tissue samples can be transferred into the three separate vessels (106, 108 and 110) and used in the age preventing compound evaluation protocol 104. The tissue samples can be a suspension of individual tissue cells that make up the tissue sample, an agglomeration of tissue cells or a tissue specimen taken from an organ/organ system.

In some embodiments, the artificial tissue samples originate from the same batch of artificially grown tissue. That is, the tissue samples transferred to each of the three vessels (106, 108 and 110) originate from the same source of tissue cells that was grown in a single batch. In other embodiments, the artificial tissue samples originate from two or more batches of artificially grown tissue. That is, the tissue samples transferred to each vessel can originate from tissue grown in different batches. It should be understood, however, that the tissue samples can be from any batch source as long as they are in substantially same physiological or cell/tissue aging condition.

After the tissue samples have been transferred, a tissue age inducing agent (e.g., doxorubicin, ionizing radiation, H₂O₂, etc.) is applied to the tissue samples in the first (‘Compound Treatment’) vessel 106 and the second (‘Positive Control Treatment’) vessel 108. In addition, a prospective age preventing agent (e.g., compound, composition, biologic, etc.) is applied to the tissue sample in the first vessel 106. In some embodiments, the tissue age inducing agent and the prospective age preventing agent are contemporaneously applied to the tissue sample in the first vessel. In other embodiments, the prospective age preventing agent is applied to the tissue sample in the first vessel 106 a set period of time before or after the tissue age inducing agent is applied to the tissue sample in the first vessel 106.

After the application of the tissue age inducing agent (the first 106 and second vessels 108) and the prospective age preventing agent (the first vessel 106 only), the tissue samples in all three vessels (the first 106, the second 108 and the third 110) are incubated for a set period of time.

Upon the completion of the incubation period, genetic material (DNA and RNA) is individually extracted 112 from the tissue samples in all three vessels (106, 108, 110), processed using an appropriate sample library preparation protocol and analyzed on a NGS (or equivalent) type of genomic sequencing system, microarray (DNA or RNA), qPCR, dPCR, etc., to measure the quantity of one or more tissue age biomarkers (e.g., RNA 114 and/or epigenetic marker 116) in each of the tissue samples.

In some embodiments, the extracted genetic material is cellular genetic material. That is, genetic material that is found within the cells comprising the tissue samples. In other embodiments, the extracted genetic material is extracellular genetic material. That is, genetic material that is either shed or secreted by the cells comprising the tissue samples.

In some embodiments, the tissue age biomarker is an epigenetic modification that is measured as a quantity of epigenetically modified DNA 116 found in the genetic material extracted from each of the tissue samples. In various embodiments, the epigenetic modification is a covalent-type DNA modification. Examples of covalent-type DNA modifications include, but are not limited to: methylation, acetylation, ubiquitylation, phosphorylation, sumoylation, ribosylation, citrullination, etc. In various embodiments, the epigenetic modification is a histone modification.

In some embodiments, the tissue age biomarker measured is a quantity of RNA 114 found in the genetic material extracted from each of the tissue samples 102. In various embodiments, the RNA measured is messenger RNA (mRNA). In various embodiments, the RNA measured is a non-coding RNA. Examples of non-coding RNA include, but are not limited to: siRNA, sRNA, microRNA, tRNA, rRNA, etc.

In still other embodiments, the quantities of a combination of different tissue age biomarkers are measured in the genetic material extracted from each of the tissue samples. For example, the quantities of both DNA methylation and mRNA can be measured in the genetic material extracted from each of the tissue samples.

Once the quantity of a tissue age biomarker has been measured for a tissue sample, the value is input into a tissue age scoring algorithm 118 which determines a score 120 that correlates with a condition (e.g., physiological/structural, functional, metabolic, etc.) that the tissue sample is in. In some embodiments, the condition is the predicted age of the tissue sample. In other embodiments, the condition is the level of cellular aging (e.g., cell damage) in the tissue sample. In still other embodiments, the condition is the level of cell death (e.g., apoptosis or necrosis) in the tissue sample. In still other embodiments, the condition is a level of proliferative cells in the tissue sample.

In some embodiments, the prospective age preventing agent is classified as an age preventing agent if the score 120 determined for the tissue sample in the first vessel 106 is less than the score 120 determined for the tissue sample in the second vessel 108. In other embodiments, the prospective age preventing agent is classified as an age-preventing agent if the score 120 determined for the tissue sample in the first vessel 106 is less than the score 120 determined for the tissue sample in the second vessel 108 and substantially similar to the score 120 determined for the tissue sample 102 in the third (‘No Treatment’) vessel 110.

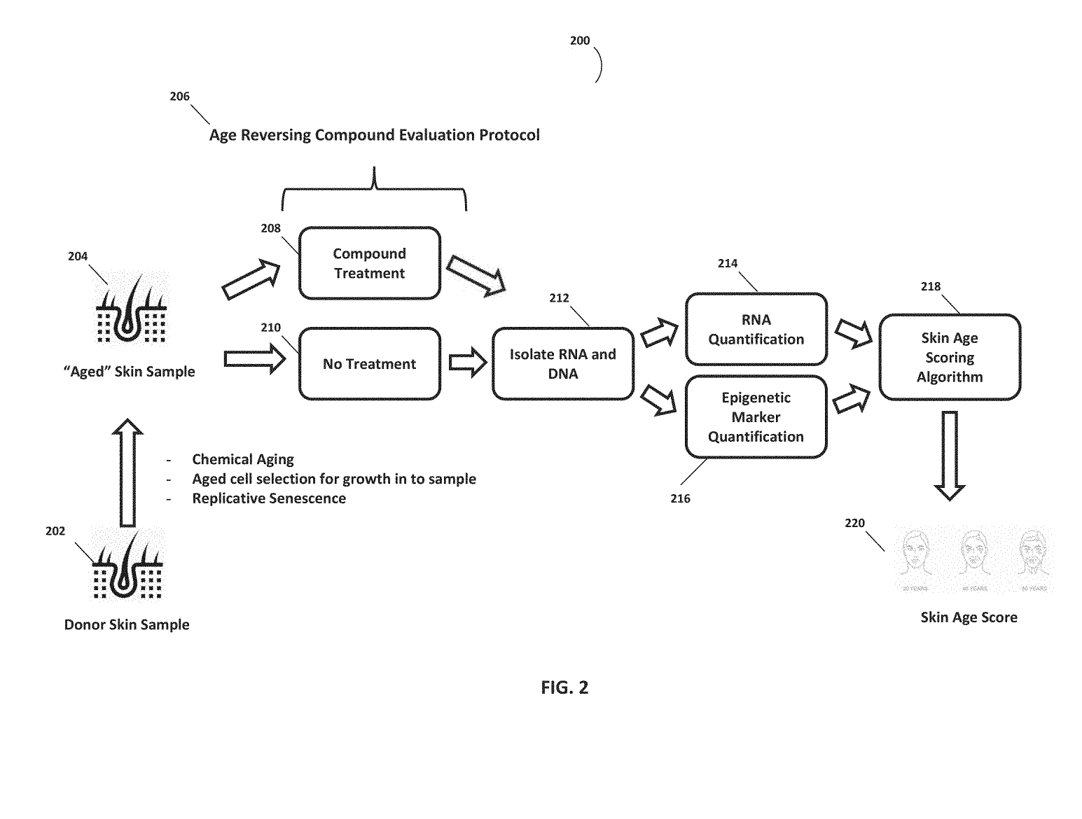

FIG. 2 illustrates a workflow for screening and discovery of skin age reversing compounds, in accordance with some embodiments of the disclosure.

As depicted herein, donor skin sample 202 is used as a starting point to produce “aged” skin sample 204. The donor skin samples 202 can be a suspension of non-tissue skin cells, an agglomeration of skin cells or a skin tissue.

In some embodiments, the donor skin sample 202 originates from the epidermis layer of the donor's skin. In other embodiments, the donor skin sample 202 originates from the dermis layer of donor's skin. In still other embodiments, the donor skin sample 202 originates from the hypodermis layer of donor's skin. That is, the donor skin sample 202 can be grown from cells or tissue grafts originating from the epidermal, dermal and/or hypodermal layers of donor's skin.

In some embodiments, a skin age inducing agent (e.g., doxorubicin, ionizing radiation, H₂O₂, etc.) is applied to donor skin sample 202 to produce an aged skin sample 204. In other embodiments, tissue engineering methodologies are employed to produce an aged skin sample 204 from donor skin sample 202. Some examples of tissue engineering techniques that can be employed include, but is not limited to: isolating individual cells that exhibit signs of aging from the donor skin sample 202 and incubating the isolated cells in conditions that promote the growth of the cells into the aged skin sample 204, incubating the donor skin sample 202 in conditions that promote cell replication (i.e., replicative senescence) to produce the aged skin sample 204, etc.

In still other embodiments, the aged skin samples 204 can be acquired directly from donors with skin that is naturally aged (i.e., elderly donors) or prematurely aged (e.g., individuals with progeria, etc.) without the need for artificial aging using a skin age inducing agent. In an exemplary embodiment, the elderly donors are greater than about 35 years of age.

After aged skin samples 204 are individually produced from donor skin samples 202 or acquired directly from a donor with naturally aged or prematurely aged skin, they are transferred to a first (‘Compound Treatment’) vessel 208 and a second (‘No Treatment’) vessel 210 and used in an age-reversing compound evaluation protocol 206. Some examples of a sample vessel may include, but are not limited to, a test tube, pipette tube, petri dish, or a well/partition within a multi-partition/well plate.

A prospective age-reversing agent (e.g., compound, composition, biologic, etc.) is then applied to the aged skin sample 204 in the first (‘Compound Treatment’) vessel 208. In some embodiments, the prospective age-reversing agent is an agent (e.g. compound, composition, biologic, etc.) that reverses cellular aging, inhibits cell apoptosis/necrosis, and/or reduces cell oxidative stress. In some exemplary embodiments, the agent is an activator of cell apoptosis/necrosis by inhibiting B-cell Lymphoma (bcl) activity.

After applying the prospective age-reversing agent (the first vessel 208 only), the aged skin sample 204 in both vessels are incubated for a set period of time. In some embodiments, the vessels are incubated for between about 24 hours to about 120 hours. In other embodiments, the vessels are incubated for between about 36 hours to about 96 hours. In still other embodiments, the vessels are incubated for between about 48 hours to about 72 hours. It should be understood, however, the vessels can essentially be incubated for as long a period of time as is necessary to determine the effectiveness of the prospective age-reversing agent.

Upon the completion of the incubation period, genetic material (DNA and RNA) is individually extracted 212 from the aged skin samples 204 in both vessels, processed using an appropriate sample library preparation protocol and analyzed on a NGS (or equivalent) type of genomic sequencing system, microarray (DNA or RNA), qPCR, dPCR, etc., to measure the quantity of one or more skin age biomarkers (e.g., RNA 214 and/or epigenetic marker 216) in each of the aged skin samples 204.

In some embodiments, the extracted genetic material is cellular genetic material. That is, genetic material that is found within the cells comprising the aged skin samples 204. In other embodiments, the extracted genetic material is extracellular genetic material. That is, genetic material that is either shed or secreted by the cells comprising the aged skin samples 204.

In some embodiments, the skin age biomarker is an epigenetic modification that is measured as a quantity of epigenetically modified DNA 216 found in the genetic material extracted from each of the aged skin samples 204. In various embodiments, the epigenetic modification is a covalent-type DNA modification. Examples of covalent-type DNA modifications include, but are not limited to: methylation, acetylation, ubiquitylation, phosphorylation, sumoylation, ribosylation, citrullination, etc. In various embodiments, the epigenetic modification is a histone modification.

In some embodiments, the skin age biomarker measured is a quantity of RNA 214 found in the genetic material extracted from each of the aged skin samples 204. In various embodiments, the RNA measured is messenger RNA (mRNA). In various embodiments, the RNA measured is a non-coding RNA. Examples of non-coding RNA include, but are not limited to: siRNA, sRNA, microRNA, tRNA, rRNA, long non-coding RNA, etc.

In still other embodiments, the quantities of a combination of different skin age biomarkers are measured in the genetic material extracted from each of the aged skin samples 204. For example, the quantities of both DNA methylation and mRNA can be measured in the genetic material extracted from each of the aged skin samples 204.

Once the quantity of a skin age biomarker has been determined for an aged skin sample 204, the value is input into a skin age scoring algorithm 218 which generates a score 220 that correlates with a condition (e.g., physiological/structural, functional, metabolic, etc.) that the aged skin sample 204 is in. In some embodiments, the condition is the predicted age of the skin sample 204. In other embodiments, the condition is the level of cellular aging (e.g., cell damage) in the aged skin sample 204. In still other embodiments, the condition is the level of cell death (e.g., apoptosis or necrosis) in the aged skin sample 204. In still other embodiments, the condition is a level of proliferative cells in the aged skin sample 204.

In some embodiments, the prospective age-reversing agent is classified as an age-reversing agent if the score 220 determined for the aged skin sample 204 in the first vessel 208 is less than the score 220 determined for the aged skin sample 204 in the second vessel 210.

In view of the potential compound screening and discovery applications discussed herein, the compound discovery workflow of FIG. 2 can be modified to allow for the screening and discovery of tissue age-reversing compounds that can be used to reverse (i.e., cure) age-related diseases and conditions (e.g., AMD, dementia, atherosclerosis, cancer, etc.).

For example, any type (e.g., muscle, cartilage, fat, skin, liver, lung, neural, cancerous or diseased, etc.) of donor tissue sample can be used as a starting point to produce an “aged” tissue sample. The donor tissue sample can be a suspension of tissue cells or a tissue specimen taken from an organ/organ system.

In some embodiments, a tissue age inducing agent (e.g., doxorubicin, ionizing radiation, H₂O₂, etc.) can be applied to the donor tissue sample to produce an aged tissue sample. In other embodiments, tissue engineering methodologies can be employed to produce an aged tissue sample from donor tissue sample. Some examples of tissue engineering techniques that can be employed include, but is not limited to: isolating individual cells that exhibit signs of aging from the donor tissue sample and incubating the isolated cells in conditions that promote the growth of the cells into an aged tissue sample, incubating the donor tissue sample in conditions that promote cell replication (i.e., replicative senescence) to produce an aged tissue sample, etc.

In still other embodiments, the aged tissue samples can be acquired directly from the diseased tissue of donors with a particular age-related disease or condition (e.g., AMD, dementia, atherosclerosis, cancer, etc.) without the need for artificial aging using a tissue age inducing agent. In one embodiment, the aged tissue sample can be RPE cells obtained from a patient suffering from AMD. In another embodiment, the aged tissue sample can be obtained from brain tissue biopsied from a patient suffering from dementia. In still another embodiment, the aged tissue sample can be obtained from vascular tissue obtained from a patient suffering from atherosclerosis. In still yet another embodiment, the aged tissue sample can be obtained from a cancerous breast tissue mass biopsied from a patient suffering from breast cancer.

After aged tissue samples are individually produced from donor tissue samples or acquired directly from the diseased tissue of donors with a particular age-related disease or condition, they are transferred to a first (‘Compound Treatment’) vessel 208 and a second (‘No Treatment’) vessel 210 and used in an age-reversing compound evaluation protocol 206. Some examples of a sample vessel may include, but are not limited to, a test tube, pipette tube, petri dish, or a well/partition within a multi-partition/well plate.

A prospective age-reversing agent (e.g., compound, composition, biologic, etc.) is then applied to the aged tissue sample in the first (‘Compound Treatment’) vessel 208. In some embodiments, the prospective age-reversing agent is an agent (e.g. compound, composition, biologic, etc.) that reverses cellular aging, inhibits cell apoptosis/necrosis, and/or reduces cell oxidative stress. After applying the prospective age-reversing agent (the first vessel 208 only), the aged tissue sample in both vessels are incubated for a set period of time. It should be understood, however, the vessels can essentially be incubated for as long a period of time as is necessary to determine the effectiveness of the prospective age-reversing agent.

Upon the completion of the incubation period, genetic material (DNA and RNA) is individually extracted 212 from the aged tissue samples in both vessels, processed using an appropriate sample library preparation protocol and analyzed on a NGS (or equivalent) type of genomic sequencing system, microarray (DNA or RNA), qPCR, dPCR, etc., to measure the quantity of one or more tissue age biomarkers (e.g., RNA 214 and/or epigenetic marker 216) in each of the aged tissue samples.

In some embodiments, the extracted genetic material is cellular genetic material. That is, genetic material that is found within the cells comprising the aged tissue samples. In other embodiments, the extracted genetic material is extracellular genetic material. That is, genetic material that is either shed or secreted by the cells comprising the aged tissue samples.

In some embodiments, the tissue age biomarker is an epigenetic modification that is measured as a quantity of epigenetically modified DNA 216 found in the genetic material extracted from each of the aged tissue samples. In various embodiments, the epigenetic modification is a covalent-type DNA modification. Examples of covalent-type DNA modifications include, but are not limited to: methylation, acetylation, ubiquitylation, phosphorylation, sumoylation, ribosylation, citrullination, etc. In various embodiments, the epigenetic modification is a histone modification.

In some embodiments, the tissue age biomarker measured is a quantity of RNA 214 found in the genetic material extracted from each of the aged tissue samples. In various embodiments, the RNA measured is messenger RNA (mRNA). In various embodiments, the RNA measured is a non-coding RNA. Examples of non-coding RNA include, but are not limited to: siRNA, sRNA, microRNA, tRNA, rRNA, long non-coding RNA, etc.

In still other embodiments, the quantities of a combination of different tissue age biomarkers are measured in the genetic material extracted from each of the aged tissue samples. For example, the quantities of both DNA methylation and mRNA can be measured in the genetic material extracted from each of the aged tissue samples.

Once the quantity of a tissue age biomarker has been determined for an aged tissue sample, the value is input into a skin age scoring algorithm 218 which generates a score 220 that correlates with a condition (e.g., physiological/structural, functional, metabolic, etc.) that the aged tissue sample is in. In some embodiments, the condition is the predicted age of the tissue sample. In other embodiments, the condition is the level of cellular aging (e.g., cell damage) in the aged tissue sample. In still other embodiments, the condition is the level of cell death (e.g., apoptosis or necrosis) in the aged tissue sample. In still other embodiments, the condition is a level of proliferative cells in the aged tissue sample.

In some embodiments, the prospective age-reversing agent is classified as an age-reversing agent if the score 220 determined for the aged tissue sample in the first vessel 208 is less than the score 220 determined for the aged tissue sample in the second vessel 210

### Computer-Implemented System

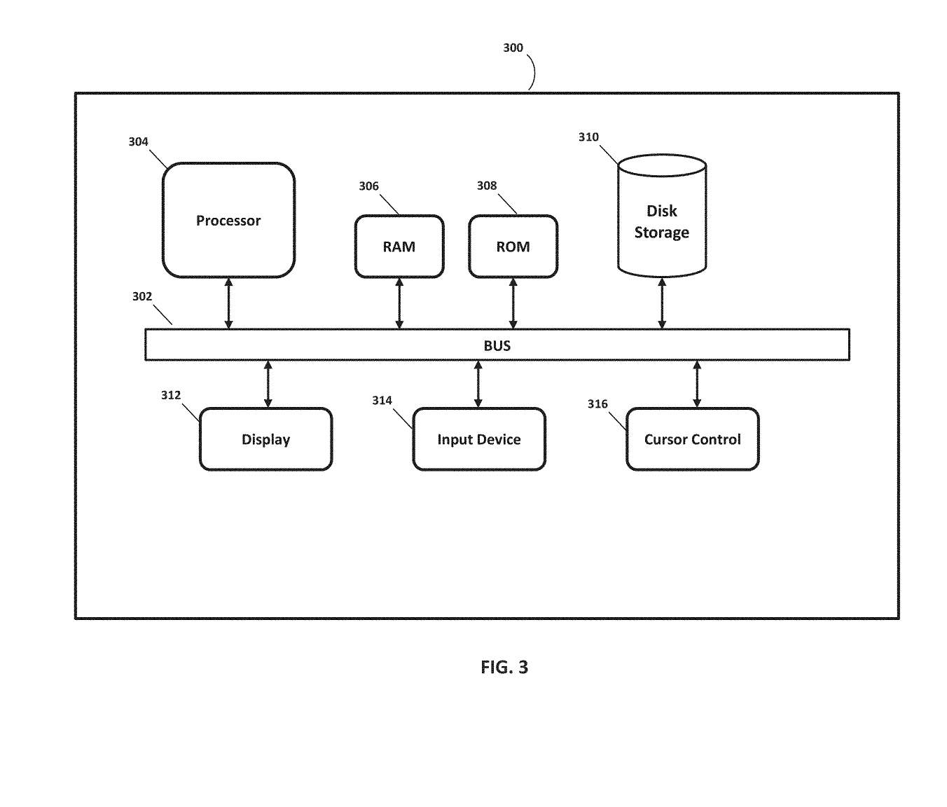

FIG. 3 is a block diagram that illustrates a computer system 300, upon which embodiments of the present teachings may be implemented. In various embodiments of the present teachings, computer system 300 can include a bus 302 or other communication mechanism for communicating information, and a processor 304 coupled with bus 302 for processing information. In various embodiments, computer system 300 can also include a memory, which can be a random access memory (RAM) 306 or other dynamic storage device, coupled to bus 302 for determining instructions to be executed by processor 304. Memory also can be used for storing temporary variables or other intermediate information during execution of instructions to be executed by processor 304. In various embodiments, computer system 300 can further include a read only memory (ROM) 308 or other static storage device coupled to bus 302 for storing static information and instructions for processor 304. A storage device 310, such as a magnetic disk or optical disk, can be provided and coupled to bus 302 for storing information and instructions.

In various embodiments, computer system 300 can be coupled via bus 302 to a display 312, such as a cathode ray tube (CRT) or liquid crystal display (LCD), for displaying information to a computer user. An input device 314, including alphanumeric and other keys, can be coupled to bus 302 for communicating information and command selections to processor 304. Another type of user input device is a cursor control 316, such as a mouse, a trackball or cursor direction keys for communicating direction information and command selections to processor 404 and for controlling cursor movement on display 312. This input device 314 typically has two degrees of freedom in two axes, a first axis (i.e., x) and a second axis (i.e., y), that allows the device to specify positions in a plane. However, it should be understood that input devices 314 allowing for 3 dimensional (x, y and z) cursor movement are also contemplated herein.

Consistent with certain implementations of the present teachings, results can be provided by computer system 300 in response to processor 304 executing one or more sequences of one or more instructions contained in memory 306. Such instructions can be read into memory 306 from another computer-readable medium or computer-readable storage medium, such as storage device 310. Execution of the sequences of instructions contained in memory 306 can cause processor 304 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 304 for execution. Such a medium can take many forms, including but not limited to, non-volatile media, volatile media, and transmission media. Examples of non-volatile media can include, but are not limited to, optical, solid state, magnetic disks, such as storage device 310. Examples of volatile media can include, but are not limited to, dynamic memory, such as memory 306. Examples of transmission media can include, but are not limited to, coaxial cables, copper wire, and fiber optics, including the wires that comprise bus 302.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 304 of computer system 300 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 300 as a standalone device or on a distributed network of shared computer processing resources such as a cloud computing network.

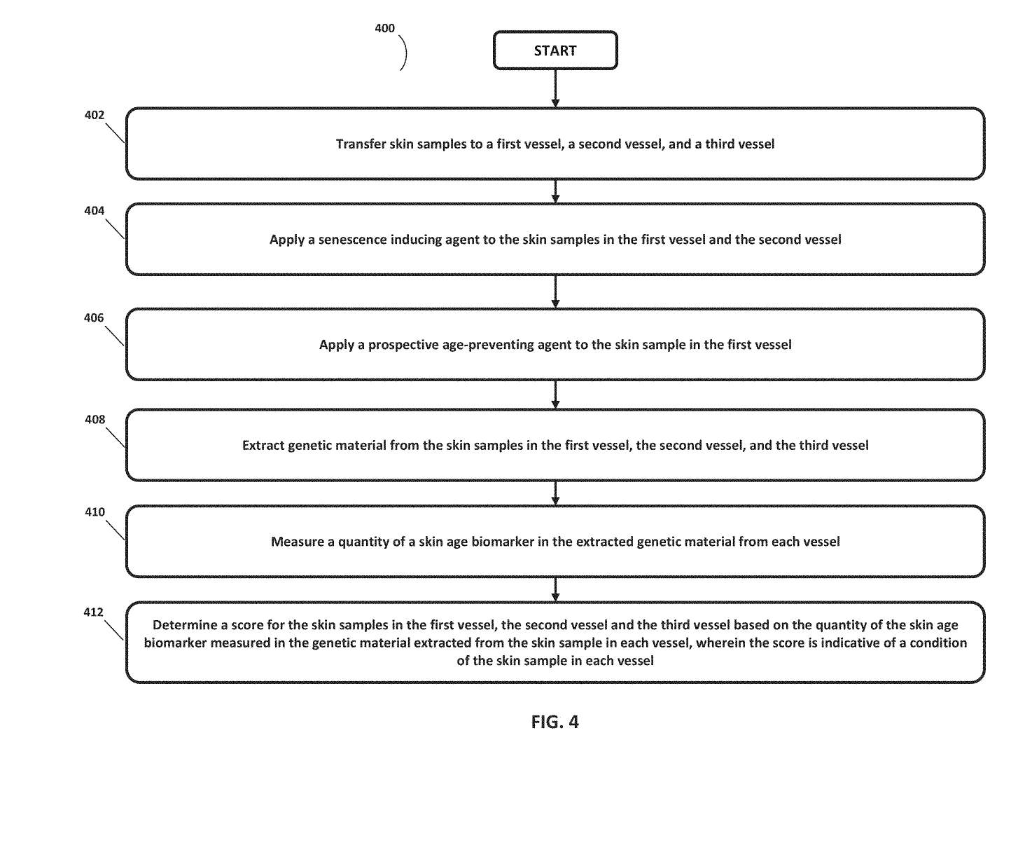

FIG. 4 is a flowchart showing a method for identifying age-preventing agents for skin, in accordance with various embodiments.

As depicted herein, method 400 details an exemplary workflow for discovering novel age-preventing agents for skin. In step 402, skin samples are transferred to a first vessel, a second vessel, and a third vessel. In some embodiments, the skin sample originates from the epidermis layer of skin. In other embodiments, the skin sample originates from the dermis layer of skin. In still other embodiments, the skin sample originates from the hypodermis layer of skin. That is, the skin sample can be grown from cells or tissue grafts originating from the epidermal, dermal and/or hypodermal layers of skin.

Some examples of a sample vessel may include, but are not limited to, a test tube, pipette tube, petri dish, or a well/partition within a multi-partition/well plate.

In some embodiments, the artificial grown skin sample originates from the same batch of artificially grown skin. That is, the skin samples transferred to each of the three vessels originate from the same source of skin cells/tissue that was grown in a single batch. In other embodiments, the artificial skin samples originate from two or more batches of artificially grown skin. That is, the skin samples transferred to each vessel can originate from skin tissue grown in different batches. It should be understood, however, that the skin samples can be from any batch source as long as they are in substantially same physiological or cell/tissue aging condition.

In step 404, a skin age inducing agent (e.g., doxorubicin, ionizing radiation, H₂O₂, etc.) is applied to the skin samples in the first (‘Compound Treatment’) vessel and the second (‘Positive Control Treatment’) vessel.

In step 406, a prospective age-preventing agent (e.g., compound, composition, biologic, etc.) is applied to the skin sample in the first vessel. In some embodiments, the skin age inducing agent and the prospective age-preventing agent are contemporaneously applied to the skin sample in the first vessel. In other embodiments, the prospective age-preventing agent is applied to the skin sample in the first vessel a set period of time after the skin age inducing agent is applied. In some embodiments, the set period of time is between about 24 hours to about 120 hours. In other embodiments, the set period of time is between about 36 hours to about 96 hours. In still other embodiments, the set period of time is between about 48 hours to about 72 hours. It should be understood, however, that the set period of time can essentially be as long a period of time as is required to determine the effectiveness of the prospective age-preventing agent.

In some embodiments, the prospective age-preventing agent is an agent (e.g. compound, composition, biologic, etc.) that prevents cellular senescence, inhibits cell apoptosis/necrosis or reduces cell oxidative stress. In some exemplary embodiments, the agent is an activator of cell apoptosis/necrosis by inhibiting B-cell Lymphoma (bcl) activity.

After applying the skin age inducing agent (the first and second vessels) and the prospective age-preventing agent (the first vessel only), the skin samples in all three vessels (the first, the second and the third) are incubated for a set period of time. In some embodiments, the vessels are incubated for between about 24 hours to about 120 hours. In other embodiments, the vessels are incubated for between about 36 hours to about 96 hours. In still other embodiments, the vessels are incubated for between about 48 hours to about 72 hours. It should be understood, however, the vessels can essentially be incubated for as long a period of time as is necessary to determine the effectiveness of the prospective age-preventing agent.

In step 408, genetic material (DNA and RNA) is extracted from the skin samples in the first vessel, the second vessel and the third vessel. In some embodiments, the extracted genetic material is cellular genetic material. That is, genetic material that is found within the cells comprising the skin samples. In other embodiments, the extracted genetic material is extracellular genetic material. That is, genetic material that is either shed or secreted by the cells comprising the skin samples.

In step 410, the quantity of a skin age biomarker is measured in the extracted genetic material. That is, after being processed using an appropriate sample library preparation protocol, the extracted genetic material is analyzed on a NGS (or equivalent) genomic sequencing system, microarray (DNA or RNA), qPCR, dPCR, etc., to measure the quantity of one or more skin age biomarkers (e.g., RNA and/or epigenetic marker, etc.) in each of the skin samples.

In some embodiments, the skin age biomarker is an epigenetic modification that is measured as a quantity of epigenetically modified DNA found in the genetic material extracted from each of the skin samples. In various embodiments, the epigenetic modification is a covalent-type DNA modification. Examples of covalent-type DNA modifications include, but are not limited to: methylation, acetylation, ubiquitylation, phosphorylation, sumoylation, ribosylation, citrullination, etc. In various embodiments, the epigenetic modification is a histone modification.

In some embodiments, the skin age biomarker measured is a quantity of RNA found in the genetic material extracted from each of the skin samples. In various embodiments, the RNA measured is messenger RNA (mRNA). In various embodiments, the RNA measured is a non-coding RNA. Examples of non-coding RNA include, but are not limited to: siRNA, sRNA, microRNA, tRNA, rRNA, long non-coding RNA, etc.

In still other embodiments, the quantities of a combination of different skin age biomarkers are measured in the genetic material extracted from each of the skin samples. For example, the quantities of both DNA methylation and mRNA can be measured in the genetic material extracted from each of the skin samples.

In step 412, a score is determined for the skin samples in the first vessel, the second vessel and the third vessel based on the quantity of the skin age biomarker measured in the genetic material extracted from the skin sample in each vessel. The score is generated by a skin age scoring algorithm using the skin age biomarker quantity values of measured for each skin sample and is indicative of a condition (e.g., physiological/structural, functional, metabolic, etc.) of the skin sample in each vessel.

In some embodiments, the condition is the predicted age of the skin sample. In other embodiments, the condition is the level of cellular aging or senescence (e.g., cell damage) in the skin sample. In still other embodiments, the condition is the level of cell death (e.g., apoptosis or necrosis) in the skin sample. In still other embodiments, the condition is a level of proliferative cells in the skin sample.

In some embodiments, the prospective age-preventing agent is classified as an age-preventing agent if the score determined for the skin sample in the first vessel is less than the score determined for the skin sample in the second vessel. In another embodiment, the prospective age-preventing agent is classified as an age-preventing agent if the score determined for the skin sample in the first vessel is less than the score determined for the skin sample in the second vessel and substantially similar to the score determined for the skin sample in the third (‘No Treatment’) vessel.

In view of the potential compound screening and discovery applications discussed herein, the compound discovery method depicted in the flowchart in FIG. 4 can be modified to allow for the screening and discovery of tissue age-preventing compounds that can be used to prevent age-related diseases and conditions (AMD, dementia, atherosclerosis, cancer, etc.).

For example, other types (e.g., muscle, cartilage, fat, skin, liver, lung, neural/brain, cancerous or diseased, etc.) of artificially grown tissue samples can be substituted in place of skin samples and processed using the method steps described in the flowchart of FIG. 4 and specification herein to screen for and discover tissue age-preventing compounds.

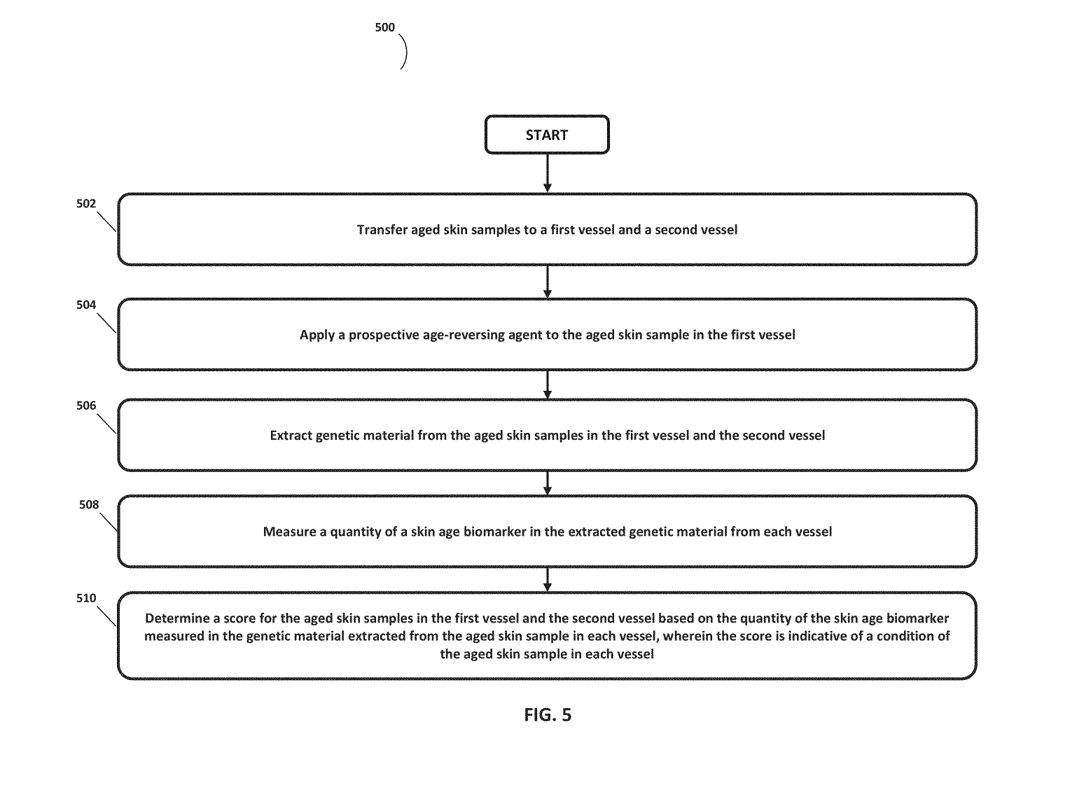

FIG. 5 is a flowchart showing a method for identifying age-reversing agents for skin, in accordance with various embodiments.

As depicted herein, method 500 details an exemplary workflow for discovering novel age-reversing agents for skin. In step 502, aged skin samples are transferred to a first vessel (‘Compound Treatment’) and a second vessel (‘No Treatment’). Some examples of a sample vessel may include, but are not limited to, a test tube, pipette tube, petri dish, or a well/partition within a multi-partition/well plate.

In various embodiments, a donor skin sample is used as a starting point to produce “aged” skin sample. The donor skin sample can be a suspension of non-tissue skin cells, an agglomeration of skin cells or skin tissue.

In some embodiments, the donor skin sample originates from the epidermis layer of the donor's skin. In other embodiments, the donor skin sample originates from the dermis layer of donor's skin. In still other embodiments, the donor skin sample originates from the hypodermis layer of donor's skin. That is, the donor skin sample can be grown from cells or tissue grafts originating from the epidermal, dermal and/or hypodermal layers of donor's skin.

In some embodiments, a skin age inducing agent (e.g., doxorubicin, ionizing radiation, H₂O₂, etc.) is applied to donor skin sample to produce an aged skin sample. In other embodiments, tissue engineering methodologies are employed to produce an aged skin sample from donor skin sample. Some examples of tissue engineering techniques that can be employed include, but is not limited to: isolating individual cells that exhibit signs of aging from the donor skin sample and incubating the isolated cells in conditions that promote the growth of the cells into the aged skin sample, incubating the donor skin sample in conditions that promote cell replication (i.e., replicative senescence) to produce the aged skin sample, etc.

In still other embodiments, the aged skin samples can be acquired directly from donors with skin that is naturally aged (i.e., elderly donors) or prematurely aged (e.g., individuals with progeria, etc.) without the need for artificial aging using a skin age inducing agent.

In step 504 a prospective age-reversing agent is applied to the aged skin sample in the first vessel. In some embodiments, the prospective age-reversing agent is an agent (e.g. compound, composition, biologic, etc.) that reverses cellular senescence, inhibits cell apoptosis/necrosis, and/or reduces cell oxidative stress. In some exemplary embodiments, the agent is an activator of cell apoptosis/necrosis by inhibiting B-cell Lymphoma (bcl) activity.

After applying the prospective age-reversing agent (the first vessel only), the aged skin samples in both vessels is incubated for a set period of time. In some embodiments, the vessels are incubated for between about 24 hours to about 120 hours. In other embodiments, the vessels are incubated for between about 36 hours to about 96 hours. In still other embodiments, the vessels are incubated for between about 48 hours to about 72 hours. It should be understood, however, the vessels can essentially be incubated for as long a period of time as is necessary to determine the effectiveness of the prospective age-reversing agent.

In step 506, genetic material is extracted from the aged skin samples in the first vessel and the second vessel. In some embodiments, the extracted genetic material is cellular genetic material. That is, genetic material that is found within the cells comprising the aged skin samples. In other embodiments, the extracted genetic material is extracellular genetic material. That is, genetic material that is either shed or secreted by the cells comprising the aged skin samples.

In step 508, a quantity of a skin age biomarker is measured in the extracted genetic material from each vessel. That is, after being processed using an appropriate sample library preparation protocol, the extracted genetic material is analyzed on a NGS (or equivalent) genomic sequencing system, microarray (DNA or RNA), qPCR, dPCR, etc., the quantity of one or more skin age biomarkers (e.g., RNA and/or epigenetic marker) in each of the aged skin samples.

In some embodiments, the skin age biomarker is an epigenetic modification that is measured as a quantity of epigenetically modified DNA found in the genetic material extracted from each of the aged skin samples. In various embodiments, the epigenetic modification is a covalent-type DNA modification. Examples of covalent-type DNA modifications include, but are not limited to: methylation, acetylation, ubiquitylation, phosphorylation, sumoylation, ribosylation, citrullination, etc. In various embodiments, the epigenetic modification is a histone modification.

In some embodiments, the skin age biomarker measured is a quantity of RNA found in the genetic material extracted from each of the aged skin samples. In various embodiments, the RNA measured is messenger RNA (mRNA). In various embodiments, the RNA measured is a non-coding RNA. Examples of non-coding RNA include, but are not limited to: siRNA, sRNA, microRNA, tRNA, rRNA, long non-coding RNA, etc.

In still other embodiments, the quantities of a combination of different skin age biomarkers are measured in the genetic material extracted from each of the aged skin samples. For example, the quantities of both DNA methylation and mRNA can be measured in the genetic material extracted from each of the aged skin samples.

In step 510, a score is determined for the skin samples in the first vessel and the second vessel based on the quantity of the skin age biomarker measured in the genetic material extracted from the aged skin sample in each vessel. The score is generated by a skin age scoring algorithm that correlates with a condition (e.g., physiological/structural, functional, metabolic, etc.) that the aged skin sample is in.

In some embodiments, the condition is the predicted age of the aged skin sample. In other embodiments, the condition is the level of cellular aging (e.g., cell damage) in the aged skin sample. In still other embodiments, the physiological condition is the level of cell death (e.g., apoptosis or necrosis) in the aged skin sample. In still other embodiments, the condition is a level of proliferative cells in the aged skin sample.

In some embodiments, the prospective age-reversing agent is classified as an age-reversing agent if the score determined for the aged skin sample in the first vessel is less than the score determined for the aged skin sample in the second vessel.

In view of the potential compound screening and discovery applications discussed herein, the compound discovery method depicted in the flowchart in FIG. 5 can be modified to allow for the screening and discovery of tissue age-reversing compounds that can be used to reverse (i.e., cure) age-related diseases and conditions (AMD, dementia, atherosclerosis, cancer, etc.).

For example, other types (e.g., muscle, cartilage, fat, skin, liver, lung, neural/brain, cancerous or diseased, etc.) of tissue can be substituted in place of skin tissue and processed using the method steps described in the flowchart of FIG. 5 and specification herein to screen for and discover tissue age-reversing compounds.

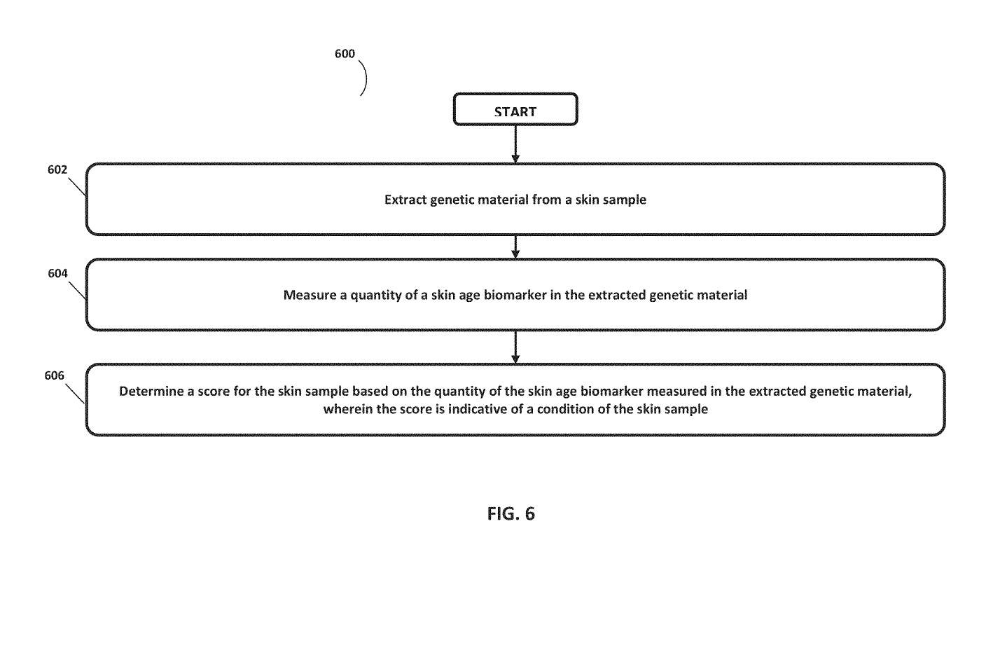

FIG. 6 is a flowchart showing a method for predicting skin age, in accordance with various embodiments.

In step 602, genetic material is extracted from a skin sample. In some embodiments, the skin sample originates from the epidermis layer of skin. In other embodiments, the skin sample originates from the dermis layer of skin. In still other embodiments, the skin sample originates from the hypodermis layer of skin. That is, the artificially grown skin sample can be grown from cells or tissue grafts originating from the epidermal, dermal and/or hypodermal layers of skin.

In step 604, a quantity of a skin age biomarker is measured in the extracted genetic material. That is, after being processed using an appropriate sample library preparation protocol, the extracted genetic material is analyzed on a NGS (or equivalent) genomic sequencing system, microarray (DNA or RNA), qPCR, dPCR, etc., the quantity of one or more skin age biomarkers (e.g., RNA and/or epigenetic marker) in the skin sample.

In some embodiments, the skin age biomarker is an epigenetic modification that is measured as a quantity of epigenetically modified DNA found in the genetic material extracted. In various embodiments, the epigenetic modification is a covalent-type DNA modification. Examples of covalent-type DNA modifications include, but are not limited to: methylation, acetylation, ubiquitylation, phosphorylation, sumoylation, ribosylation, citrullination, etc. In various embodiments, the epigenetic modification is a histone modification.

In some embodiments, the skin age biomarker measured is a quantity of RNA found in the genetic material extracted from each of the aged skin samples. In various embodiments, the RNA measured is messenger RNA (mRNA). In various embodiments, the RNA measured is a non-coding RNA. Examples of non-coding RNA include, but are not limited to: siRNA, sRNA, microRNA, tRNA, rRNA, etc. Measurement of RNA can be carried out using routine methods for nucleic acid analysis, e.g., Northern blot, DNA microarray, sequencing, etc. Preferably, RNA analysis is carried out using gene expression assays.

In still other embodiments, the quantities of a combination of different skin age biomarkers are measured in the genetic material extracted from each of the aged skin samples. For example, the quantities of both DNA methylation and mRNA can be measured in the genetic material extracted from each of the aged skin samples.

In step 606, a score is determined for the skin sample based on the quantity of the skin age biomarker measured in the genetic material extracted from the skin sample. The score is generated by a skin age scoring algorithm that correlates with a condition (e.g., physiological/structural, functional, metabolic, etc.) that the skin sample is in.

In some embodiments, the condition is the predicted age of the skin sample. In other embodiments, the condition is the level of cellular aging (e.g., cell damage) in the skin sample. In still other embodiments, the physiological condition is the level of cell death (e.g., apoptosis or necrosis) in the skin sample. In still other embodiments, the condition is a level of proliferative cells in the skin sample.

It should be appreciated, however, that the method described in the flowchart in FIG. 6 can also be applied to predict the age of non-skin tissue. For example, other types (e.g., muscle, cartilage, fat, skin, liver, lung, neural/brain, cancerous or diseased, etc.) of tissue can be substituted in place of skin tissue and processed using the method steps described in the flowchart of FIG. 6 and specification herein so that the age of the tissue can be predicted.

Some of the methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400 of FIG. 4, whereby processor 404 would execute the analyses and determinations provided by these engines, subject to instructions provided by any one of, or a combination of, memory components 406/4008/410 and user input provided via input device 414.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical, FLASH memory and non-optical data storage devices. The computer readable medium can also be distributed over a network coupled computer systems so that the computer readable code is stored and executed in a distributed fashion.

Markers

The methods for screening compounds (e.g., anti-aging compounds) of the disclosure preferably include one or more genetic markers. The genetic markers include genes, gene expression products (mRNA or protein). Preferably, the genetic markers include changes in expression of one or more of the genes in Table 1.

TABLE 1
List of markers associated with aging

| | | | ENTREZ | NCBI | |
|---|---|---|---|---|---|
| | | | Accession | Accession | Correlation |
| Tissue | Marker | Full name | Gene | mRNA | w/aging |
| Skin | P16 | cyclin-dependent kinase | 1029 | NM_000077; | Positive |
| | | inhibitor 2A, multiple tumor | | NM_001195132; | |
| | | suppressor 1 | | NM_058195; | |
| | | | | NM_058196; | |
| | | | | NM_058197 | |
| Skin | IL8 | Interleukin 8 | 3576 | NM_000584; | Positive |
| | | | | NM_001354840 | |
| Skin | MMP-1 | Matrix metalloproteinase-1 | 4312 | NM_002421; | Positive |
| | | | | NM_001145938 | |
| Skin | HAS-2 | Hyaluronan synthase 2 | 3037 | NM_005328 | Negative |
| Skin | ZIC1 | Zinc finger of the | 7545 | NM_003412 | Positive |
| | | cerebellum 1 | | | |
| Skin | BLIMP1 | PR domain zinc finger | 639 | NM_001198; | Positive |
| | | protein 1 | | NM_182907 | |
| Skin | KI67 | Antigen KI-67 | 4288 | NM_001145966; | Negative |
| | | | | NM_002417 | |
| Skin | ZYG11B | Zyg-11 family member B, | 79699 | NM_024646 | Positive |
| | | cell cycle regulator | | | |

Preferably, the markers include at least 1, 2, 3, 4, 5, 6, 7 or 8 of the markers of Table 1.

Especially, the markers include at least 1, 2, or 3 of the following markers: ZIC1, BLIMP1 or ZYG11B.

In some embodiments, the markers include 1, 2, or 3 of the following markers: ZIC1, BLIMP1 or ZYG11B and at least 1, 2, 3, 4, or 5 of the following markers: P16; IL8; MMP-1; HAS-2; and/or KI67.

In some embodiments, the markers include signatures, e.g., signature 1 comprising at least 1, 2, 3, 4, or 5 of the following genes: P16; IL8; MMP-1; HAS-2; and/or ZIC1; signature 2 comprising at least 1, 2, 3, 4, or 5 of the following genes: P16; IL8; BLIMP1; KI67 and ZYG11B; signature 3 comprising at least 1, 2, 3, 4, 5, or 6 positively correlated genes comprising P16; IL8; MMP-1; ZIC1; BLIMP1 and/or ZYG11B or signature 4 comprising at least 1 or 2 negatively correlated genes comprising HAS2 and/or KI67.

In some embodiments, the markers are weighed differently with respect to the aging parameter being measured. For instance, in aforementioned signature 1, the order of weighing (from high to low) is as follows: P16, ZIC-1, MMP-1, HAS-2, and IL-8. Especially, ZIC-1 is included in such signatures as it correlated very strongly with aging. Similarly, in aforementioned signature 2, the order of weighing (from high to low) is as follows: BLIMP-1, P16, ZYG11B, IL-8, and KI-67. Especially, BLIMP-1 and/or ZYG11B are included in such signatures as they both correlated very strongly with aging.

In some embodiments, wherein the tissue is dermis, the genetic markers include genes or gene expression products (mRNA or protein) of Table 2. Preferably, the genetic markers include changes in expression of one or more of the genes in Table 2.

TABLE 2
List of markers associated with aging in dermis

| | | | ENTREZ | NCBI | |
|---|---|---|---|---|---|
| | | | Accession | Accession | Correlation |
| Tissue | Marker | Full name | Gene | mRNA | w/aging |
| Dermis | P16 | cyclin-dependent kinase | 1029 | NM_000077; | Positive |
| | | inhibitor 2A, multiple tumor | | NM_001195132; | |
| | | suppressor 1 | | NM_058195; | |
| | | | | NM_058196; | |
| | | | | NM_058197 | |
| Dermis | IL8 | Interleukin 8 | 3576 | NM_000584; | Positive |
| | | | | NM_001354840 | |
| Dermis | MMP-1 | Matrix metalloproteinase 1 | 4312 | NM_002421; | Positive |
| | | | | NM_001145938 | |
| Dermis | HAS-2 | Hyaluronan synthase 2 | 3037 | NM_005328 | Negative |
| Dermis | ZIC1 | Zinc finger of the | 7545 | NM_003412 | Positive |
| | | cerebellum 1 | | | |

Preferably, the markers include at least 1, 2, 3, 4, or 5 of the markers of Table 2.

Especially, the markers of Table 2 include at least ZIC1.

In some embodiments, the markers include ZIC1 and at least 1, 2, 3, or 4 of the following markers: P16; IL8; MMP-1; and/or HAS-2.

In some embodiments, the markers include signatures, e.g., signature 1 comprising at least 1, 2, 3, 4, or 5 of the following genes: P16; IL8; MMP-1; HAS-2; and/or ZIC1; or signature 2 comprising positively correlated genes comprising P16; IL8; MMP-1; and ZIC1.

In some embodiments, the markers are weighed differently with respect to the aging parameter being measured. For instance, in aforementioned signature, the order of weighing (from high to low) is as follows: P16, ZIC-1, MMP-1, HAS-2, and 11-8. Especially, ZIC-1 is included in such signatures as it correlated very strongly with aging in dermis tissue.

In some embodiments, wherein the tissue is epidermis, the genetic markers include genes or gene expression products (mRNA or protein) of Table 3. Preferably, the genetic markers include changes in expression of one or more of the genes in Table 3.

TABLE 3
List of markers associated with aging in epidermis

| | | | NCBI | NCBI | |
|---|---|---|---|---|---|
| | | | Accession | Accession | Correlation |
| Tissue | Marker | Full name | Gene | mRNA | w/aging |
| Epidermis | P16 | cyclin-dependent kinase | 1029 | NM_000077; | Positive |
| | | inhibitor 2A, multiple tumor | | NM_001195132; | |
| | | suppressor 1 | | NM_058195; | |
| | | | | NM_058196; | |
| | | | | NM_058197 | |
| Epidermis | IL8 | Interleukin 8 | 3576 | NM_000584; | Positive |
| | | | | NM_001354840 | |
| Epidermis | BLIMP1 | PR domain zinc finger | 639 | NM_001198; | Positive |
| | | protein 1 | | NM_182907 | |
| Epidermis | KI67 | Antigen KI-67 | 4288 | NM_001145966; | Negative |
| | | | | NM_002417 | |
| Epidermis | ZYG11B | Zyg-11 family member B, | 79699 | NM_024646 | Positive |
| | | cell cycle regulator | | | |

Preferably, the markers include at least 1, 2, 3, 4, or 5 of the markers of Table 3.

Especially, the markers of Table 3 include at least ZYG11B or BLIMP1 or both ZYG11B and BLIMP1.

In some embodiments, the markers include ZYG11B or BLIMP1 or both ZYG11B and BLIMP1 and at least 1, 2, or 3 of the following markers: P16; IL8; and/or KI67.

In some embodiments, the markers include signatures, e.g., signature 1 comprising at least 1, 2, 3, 4, or 5 of the following genes: ZYG11B; BLIMP1; P16; IL8; and/or KI67; or signature 2 comprising positively correlated genes comprising P16; IL8; ZYG11B; and/or BLIMP1.

In some embodiments, the markers are weighed differently with respect to the aging parameter being measured. For instance, in aforementioned signature, the order of weighing (from high to low) is as follows: BLIMP-1, P16, ZYG11B, IL-8, and KI-67. Especially, BLIMP-1 and/or ZYG11B are included in such signatures as they both correlated very strongly with aging in epidermis tissue.

High Throughput Screening

In accordance with above-described methods, the instant disclosure relates to high throughput screening (HTS) methods. Herein, a small-molecule drug discovery project usually begins with screening a large collection of compounds against a biological target that is believed to be associated with a certain disease. The goal of such screening is generally to identify interesting, tractable starting points for medicinal chemistry. Despite the fact that screening of huge libraries containing as many as one million compounds can now be accomplished in a matter of days in pharmaceutical companies, the number of compounds that eventually enter the medicinal chemistry phase of lead optimization is still largely limited to a couple of hundred compounds at best. In that regard, it is generally well understood that one significant challenge to the early hit-to-lead process of drug discovery is selecting the most promising compounds from primary HTS results.

In current HTS data analysis, an activity cutoff value is usually set to allow selection of a certain number of compounds whose tested activities are greater than (or less than, depending upon the application) this threshold. The selected compounds are called “primary hits” and are subject to retesting for confirmation. Following such retesting and confirmation, confirmed or validated primary hit compounds are grouped into families. Based upon further evaluation or additional chemical exploration, the families that exhibit certain desired or promising characteristics (such as, for example, a certain degree of structure-activity relationship (SAR) among the compounds in the family, advantageous patent status, amenability to chemical modification, favorable physicochemical and pharmacokinetic properties, and so forth) are selected as lead series for subsequent analysis and optimization.

In accordance with some embodiments, for example, a high-throughput screening hit identification method may generally comprise: selecting a family of compounds to be analyzed; evaluating the family of compounds in accordance with a relationship characteristic; and prioritizing ones of the compounds in accordance with evaluation methodology of the disclosure (e.g., analyzing changes in expression, levels, or activities of the biomarkers of the disclosure). Some such methods may further comprise selectively repeating the selecting and the evaluating until a predetermined number of families of compounds has been selected and evaluated.

Embodiments are disclosed wherein the evaluating comprises assigning a probability score to the family of compounds; such assigning may comprise, for example, computing a non-parametric probability score, calculating the probability score based upon an hypergeometric probability distribution, or both. The evaluating may be executed in accordance with a structure-activity relationship analysis, for instance, or in accordance with a mechanism-activity relationship. Some exemplary methods for evaluation of screened compounds comprise ranking the compounds in accordance with an activity criterion; in methods employing such ranking, the prioritizing may further comprise analyzing selected ones of the compounds in accordance with the ranking and the evaluating.

In some embodiments, a computer-readable medium encoded with data and instructions for high-throughput screening hit selection may be used. The data and instructions may cause an apparatus executing the instructions to: identify a family of compounds to be analyzed; rank each respective compound to be analyzed with respect to an activity criterion (e.g., changes in levels or activity of one of the markers of Table 1 or a product thereof); evaluate the family of compounds in accordance with a relationship characteristic; and prioritize ones of the compounds in accordance with results of the evaluation and in accordance with rank.

The computer-readable medium may be further encoded with data and instructions causing an apparatus executing the instructions selectively to repeat identifying a family of compounds and evaluating the family of compounds. In some embodiments, the data and instructions may further cause an apparatus executing the instructions to assign a probability score to the family of compounds; as set forth below, this may involve computing a non-parametric probability score, calculating the probability score based upon an hypergeometric probability distribution, or both.

For some applications, the computer-readable medium may be further encoded with data and instructions causing an apparatus executing the instructions to evaluate the family of compounds in accordance with a structure-activity relationship analysis or in accordance with a mechanism-activity relationship analysis.

In some implementations, an exemplary high-throughput screening system may generally comprise: a processor operative to execute data processing operations; a memory encoded with data and instructions accessible by the processor; and a hit selector operative, in cooperation with the processor, to: identify a family of compounds to be analyzed; evaluate the family of compounds in accordance with a relationship characteristic; and prioritize ones of the compounds in accordance with results of the evaluation and in accordance with a rank for each respective compound, the rank being associated with an activity criterion.

Embodiments are disclosed wherein the hit selector is further operative selectively to repeat identifying a family of compounds and evaluating the family of compounds. The hit selector may be further operative to assign a probability score to the family of compounds.

In some systems, the hit selector is further operative to evaluate the family of compounds in accordance with a structure-activity relationship analysis; additionally or alternatively, the hit selector may be further operative to evaluate the family of compounds in accordance with a mechanism-activity relationship analysis.

### EXAMPLES

The structures, materials, compositions, and methods described herein are intended to be representative examples of the disclosure, and it will be understood that the scope of the disclosure is not limited by the scope of the examples. Those skilled in the art will recognize that the disclosure may be practiced with variations on the disclosed structures, materials, compositions and methods, and such variations are regarded as within the ambit of the disclosure.

### Example 1

### Gene Expression in Skin Samples

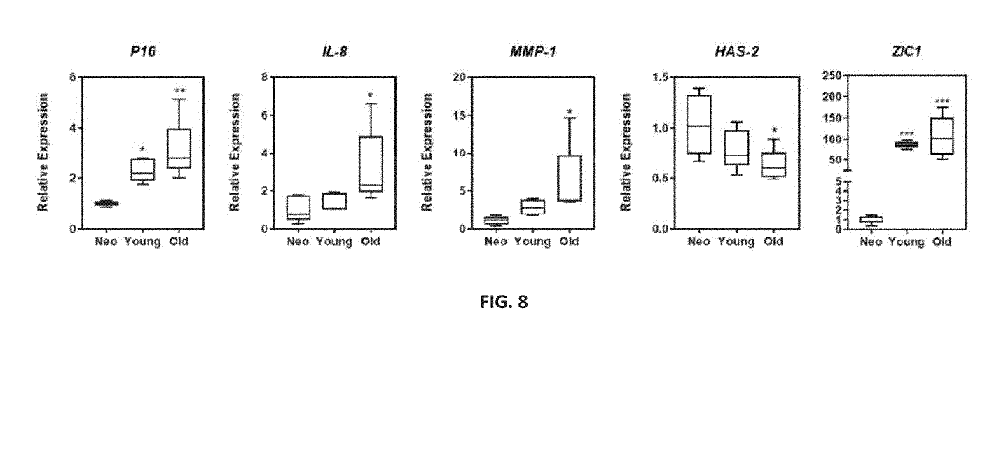

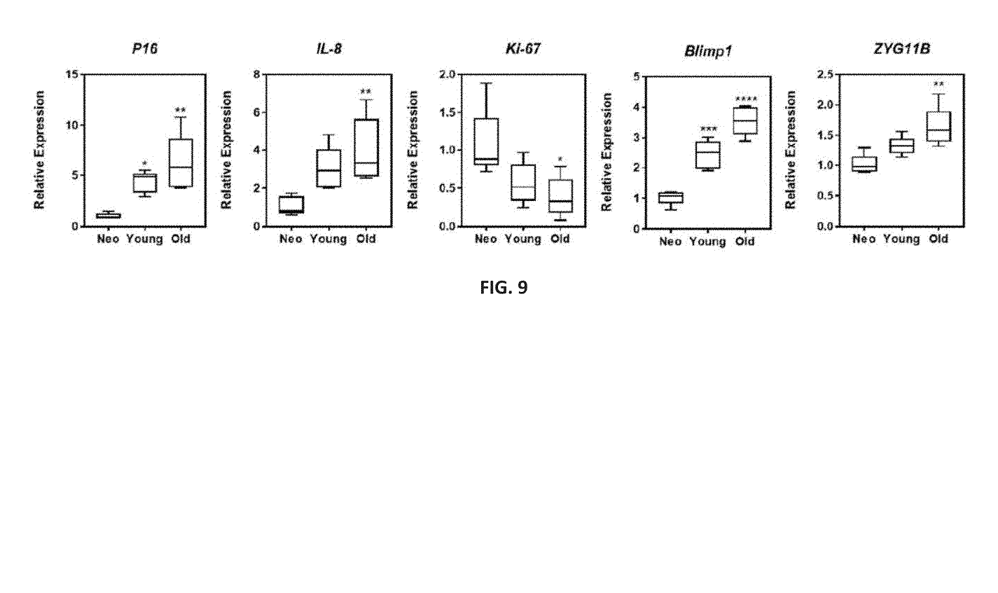

Skin tissues were built on air liquid grids. They were cultured for about 12 days, during which media is changed every 2-3 days. Tissues are grouped as follows: Groups Neo—Neonatal Fb, Neonatal Kc; 29y—29 year old Fb, 60y Kc; 84y—84 year old Fb, 60y Kc. At the end of the period, gene expression analysis is conducted. Data are shown in FIG. 8 and FIG. 9. With respect to gene expression in the dermis, the results, which are presented in FIG. 8, show that expression of the following genes is upregulated in an age-dependent manner: P16, IL-8, MMP-1, and ZIC-1. In contrast, HAS-2 expression decreases with age. The correlation of ZIC-1 with aging dermal skin tissue was hitherto unknown, which has been illustrated for the first time by these studies. With respect to gene expression in the epidermis, the results, which are presented in FIG. 9, show that the expression of the following genes is upregulated in an age-dependent manner: P16, IL-8, Blimp-1, and ZYG11B. In contrast, Ki-67 expression decreases with age. The correlation of Blimp-1, and ZYG11B with aging dermal skin tissue was hitherto unknown, which has been illustrated for the first time by these studies.

### Example 2

### Building a Machine Learning Model to Screen and/or Test Compounds

Analysis of the expression of the selected genes, e.g., ZIC-1 optionally together with P16, IL-8, MMP-1, and HAS-2 in the context of dermal tissue and/or Blimp-1 and/or ZYG11B optionally together with P16, IL-8, and Ki-67 in the context of epidermal tissue, were used to build a Machine Learning model that allows to quantify the effect of compounds on the skin regarding their ability to prevent, halt or reverse aging. The model was created as follows:

Training of the Model

Real Time PCR was performed on the target genes. All values are normalized by the housekeeping gene only, and we assume that between experiments the housekeeping genes are behaving similar (the parameter used is DeltaCT instead of Delta_DeltaCT). Next, Machine Learning models were created based on the Poisson regression algorithm for Dermis and a Deep Learning model based on the Neural Network algorithm for Epidermis. The models were evaluated using Root Mean Squared Error (RMSE), which is the value of the average distance of the residuals from zero and is calculated by taking the square root of the mean squared error (MSE). The smaller the value, the better because they provide more information. Also, Mean Absolute Error (MAE) values were computed. Here too, the smaller the value the better. Coefficient of Determination (R²) values were further computed, wherein R² values approaching 1 are regarded to be better. The model was built using the Microsoft tool called Azure ML Studio with the following parameters: Tune Model hyperparameters; Cross Validation 10×. About 80% of the dataset was used for training the model.

It was also found that in the context of dermis tissue, the following genes had significant Pearson correlation coefficient (all >0.5) with age, wherein the genes are listed in the order from higher to lower correlation: P16, ZIC-1, MMP-1, HAS-2, 11-8. The coefficient of determination (R²) of the model was 0.96, which represents a very high association. Particularly, ZIC-1 correlated very strongly with aging using the Pearson correlation. In various embodiments, these correlations can be ascertained using machine learning or artificial intelligence models such as, but not limited to, Poisson distribution model or a neural network model.

In the context of epidermis, based on a neural network model, it was found that the following genes had significant Pearson correlation coefficient (all >0.5) with age, wherein the genes are listed in the order from higher to lower correlation: Blimp-1, P16, ZYG11B, 11-8, and Ki-67. Particularly, Blimp-1 and ZYG11B both correlated very strongly with aging. In various embodiments, these correlations can be ascertained using machine learning or artificial intelligence models such as, but not limited to, Poisson distribution model or a neural network model.

Testing Compounds:

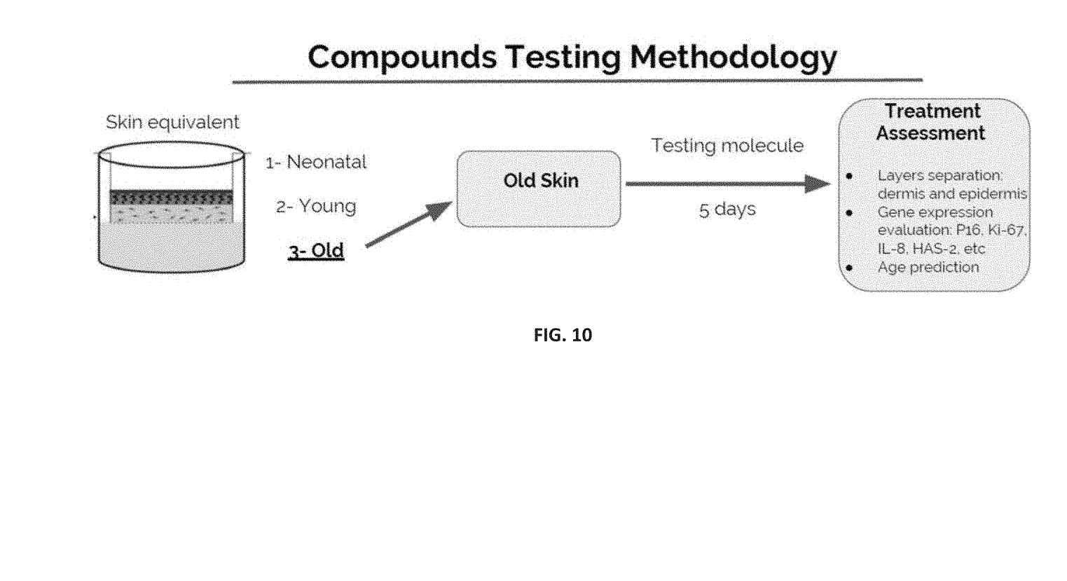

A schematic diagram for testing compounds is shown in FIG. 10. Skin tissues were built on air liquid grids and cultured as described above. Test molecules were added on old (89 year-old) skin tissues, and the tissues were cultured for 5 days. To assess whether the test compounds were effective, dermis and epidermis layers were separated, gene expression evaluation was conducted using real-time PCR (rtPCR) to analyze the aforementioned genes (dermis: P16, 11-8, MMP-1, HAS-2 and ZIC1; and epidermis: P16, 11-8, Blimp1, Ki67 and ZYG11B). Samples were treated with two test compounds (A and B) and a positive control (retinoic acid, which increases aging). Following treatment, age was predicted based on the expression levels of the marker genes. More specifically, the cycle threshold (CT) value of each gene was used in the Algorithm to predict the skin age. Results are shown in Table 4 (dermis tissue) and Table 5 (epidermis tissue).

In dermis samples, a reduction of the dermal age was observed when the skin samples were treated with the Compound 2. Treatment with Compound 1 had a small effect in lowering the age of skin. In contrast, the treatment with retinoic acid increased the skin age. Results are shown in Table 4.

TABLE 4
Model testing results in dermis

| | | Age Scored |
|---|---|---|
| | | by Dermis |
| Experiment | Tissue | model |
| Control | Dermis | 43.132 |
| Compound 1 | Dermis | 41.676 |
| Control | Dermis | 91.463 |
| Compound 2 | Dermis | 52.136 |
| Control | Dermis | 60.606 |
| 10 μM Retinoic Acid | Dermis | 80.308 |

In epidermis samples, a reduction of the dermal age was observed when the skin samples were treated with the Compound 1. Treatment with Compound 2 had a smaller, but also appreciable effect in lowering the age of skin. In contrast, the treatment with retinoic acid increased the skin age. Results are shown in Table 5.

TABLE 5
Model testing results in epidermis

| | | Age Scored |
|---|---|---|
| | | by |
| | | Epidermis |
| Experiment | Tissue | model |
| Control | Epidermis | 46.079 |
| Compound 1 | Epidermis | 12.996 |
| Control | Epidermis | 28.689 |
| Compound 2 | Epidermis | 20.258 |
| Control | Epidermis | 6.662 |
| 10 μM Retinoic Acid | Epidermis | 79.376 |

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout this disclosure, various patents, patent applications and publications are referenced. The disclosures of these patents, patent applications, accessioned information (e.g., as identified by PUBMED, PUBCHEM, NCBI, UNIPROT, or EBI accession numbers) and publications in their entireties are incorporated into this disclosure by reference in order to more fully describe the state of the art as known to thosedisclosre00skilled therein as of the date of this disclosure. This disclosure will govern in the instance that there is any inconsistency between the patents, patent applications and publications cited and this disclosure.

### Recitation of Selected Embodiments

Embodiment 1. A method for identifying age-preventing agents for skin, comprising: transferring skin samples to a first vessel, a second vessel, and a third vessel; applying a skin age inducing agent to the skin samples in the first vessel and the second vessel; applying a prospective age-preventing agent to the skin sample in the first vessel; extracting genetic material from the skin samples in the first vessel, the second vessel and the third vessel; measuring a quantity of a skin age biomarker in the extracted genetic material from each vessel; and determining a score for the skin samples in the first vessel, the second vessel and the third vessel based on the quantity of skin age biomarker measured in the genetic material extracted from the skin sample in each vessel, wherein the score is indicative of a condition of the skin sample in each vessel.

Embodiment 2. The method of Embodiment 1, wherein the skin age biomarker is RNA.

Embodiment 3. The method of Embodiment 2, wherein the RNA is a messenger RNA (mRNA).

Embodiment 4. The method of Embodiment 2, wherein the RNA is a non-coding RNA.

Embodiment 5. The method of Embodiment 4, wherein the non-coding RNA is microRNA.

Embodiment 6. The method of Embodiment 4, wherein the non-coding RNA is siRNA.

Embodiment 7. The method of Embodiment 1, wherein the skin age biomarker is an epigenetic modification.

Embodiment 8. The method of Embodiment 7, wherein the epigenetic modification is a covalent-type modification.

Embodiment 9. The method of Embodiment 8, wherein the covalent-type modification is methylation.

Embodiment 10. The method of Embodiment 7, wherein the epigenetic modification level is a histone-type modification.

Embodiment 11. The method of Embodiment 1, wherein the condition is a predicted age of the skin sample.

Embodiment 12. The method of Embodiment 1, wherein the condition is a level of cellular senescence in the skin sample.

Embodiment 13. The method of Embodiment 12, wherein the level of cell senescence is indicative of cell apoptosis in the skin sample.

Embodiment 14. The method of Embodiment 12, wherein the level of cell senescence is indicative of cell necrosis in the skin sample.

Embodiment 15. The method of Embodiment 1, wherein the condition is a level of oxidative stress in the skin sample.

Embodiment 16. The method of Embodiment 11, further including: classifying the prospective age-preventing agent as an age-preventing agent if the score determined for the skin sample in the first vessel is less than the score determined for the skin sample in the second vessel.

Embodiment 17. The method of Embodiment 1, wherein the skin age inducing agent and the prospective age-preventing agent are applied simultaneously to the skin sample in the first vessel.

Embodiment 18. A method for identifying age-reversing agents for skin, comprising: transferring aged skin samples to a first vessel and a second vessel; applying a prospective age-reversing agent to the aged skin sample in the first vessel; extracting genetic material from the aged skin samples in the first vessel and the second vessel; measuring a quantity of a skin age biomarker in the extracted genetic material from each vessel; and determining a score for the aged skin samples in the first vessel and the second vessel based on the quantity of the skin age biomarker measured in the genetic material extracted from the aged skin sample in each vessel, wherein the score is indicative of a condition of the aged skin sample in each vessel.

Embodiment 19. The method of Embodiment 18, further including: applying a skin age inducing agent to a donor skin sample to create the aged skin sample.

Embodiment 20. The method of Embodiment 18, further including: isolating aged skin cells from a donor skin sample; transferring the isolated aged skin cells to an incubation vessel; and incubating the aged skin cells under conditions that promote growth of the aged skin cells into the aged skin sample.

Embodiment 21. The method of Embodiment 20, wherein the donor skin sample is obtained from a donor of greater than about 35 years of age.

Embodiment 22. The method for Embodiment 20, wherein the donor skin sample is obtained from a donor with progeria.

Embodiment 23. The method of Embodiment 18, further including: incubating a donor skin sample under conditions that promote cell replication to create the aged skin sample.

Embodiment 24. The method of Embodiment 18, wherein the skin senescence biomarker is RNA.

Embodiment 25. The method of Embodiment 24, wherein the RNA is messenger RNA (mRNA).

Embodiment 26. The method of Embodiment 24, wherein the RNA is a non-coding RNA.

Embodiment 27. The method of Embodiment 26, wherein the non-coding RNA is siRNA.

Embodiment 28. The method of Embodiment 26, wherein the non-coding RNA is microRNA.

Embodiment 29. The method of Embodiment 18, wherein the skin age biomaker is an epigenetic modification

Embodiment 30. The method of Embodiment 29, wherein the epigenetic modification is a covalent-type modification.

Embodiment 31. The method of Embodiment 30, wherein the covalent-type modification is methylation.

Embodiment 32. The method of Embodiment 29, wherein the epigenetic modification is a histone-type modification.

Embodiment 33. The method of Embodiment 18, wherein the condition is a predicted age of the aged skin sample.

Embodiment 34. The method of Embodiment 18, wherein the condition is a level of cellular senescence in the aged skin sample.

Embodiment 35. The method of Embodiment 34, wherein the level of cell senescence is indicative of cell apoptosis in the aged skin sample.

Embodiment 36. The claim of Embodiment 34, wherein the level of cell senescence is indicative of cell necrosis in the aged skin sample.

Embodiment 37. The method of Embodiment 18, wherein the condition is a level of oxidative stress in the aged skin sample.

Embodiment 38. The method of Embodiment 18, further including: classifying the prospective age-reversing agent as an age-reversing agent if the score determined for the aged skin sample in the first vessel is less than the score determined for the aged skin sample in the second vessel.

Embodiment 39. A method for predicting skin age, comprising: extracting genetic material from a skin sample; measuring a quantity of a skin age biomarker in the extracted genetic material; and determining a score for the skin sample based on the quantity of the skin age biomarker measured in the extracted genetic material, wherein the score is indicative of a condition of the skin sample.

Embodiment 40. The method of Embodiment 39, wherein the skin age biomarker is RNA.

Embodiment 41. The method of Embodiment 40, wherein the RNA is messenger RNA (mRNA).

Embodiment 42. The method of Embodiment 39, wherein the RNA is a non-coding RNA.

Embodiment 43. The method of Embodiment 42, wherein the non-coding RNA is siRNA.

Embodiment 44. The method of Embodiment 42, wherein the non-coding RNA is microRNA.

Embodiment 45. The method of Embodiment 38, wherein the skin age biomaker is an epigenetic modification

Embodiment 46. The method of Embodiment 45, wherein the epigenetic modification is a covalent-type modification.

Embodiment 47. The method of Embodiment 46, wherein the covalent-type modification is methylation.

Embodiment 48. The method of Embodiment 45, wherein the epigenetic modification is a histone-type modification.

Embodiment 49. The method of Embodiment 39, wherein the condition is a predicted age of the skin sample.

Embodiment 50. The method Embodiment 42, wherein the non-coding RNA is a long non-coding RNA.

Embodiment 51. The method of Embodiment 19, wherein the skin age inducing agent is selected from the group consisting of doxorubicin, hydrogen peroxide and ionizing radiation.

Embodiment 52. A method for identifying age-preventing agents for tissue, comprising: transferring tissue samples to a first vessel, a second vessel, and a third vessel; applying a tissue age inducing agent to the tissue samples in the first vessel and the second vessel; applying a prospective age-preventing agent to the tissue sample in the first vessel; extracting genetic material from the tissue samples in the first vessel, the second vessel and the third vessel; measuring a quantity of a tissue age biomarker in the extracted genetic material from each vessel; and determining a score for the tissue samples in the first vessel, the second vessel and the third vessel based on the quantity of tissue age biomarker measured in the genetic material extracted from the tissue sample in each vessel, wherein the score is indicative of a condition of the tissue sample in each vessel.

Embodiment 53. The method of Embodiment 52, wherein the tissue age biomarker is RNA.

Embodiment 54. The method of Embodiment 53, wherein the RNA is a messenger RNA (mRNA).

Embodiment 55. The method of Embodiment 53, wherein the RNA is a non-coding RNA.

Embodiment 56. The method of Embodiment 55, wherein the non-coding RNA is microRNA.

Embodiment 57. The method of Embodiment 55, wherein the non-coding RNA is siRNA.

Embodiment 58. The method of Embodiment 52, wherein the tissue age biomarker is an epigenetic modification.

Embodiment 59. The method of Embodiment 58, wherein the epigenetic modification is a covalent-type modification.

Embodiment 60. The method of Embodiment 59, wherein the covalent-type modification is methylation.

Embodiment 61. The method of Embodiment 58, wherein the epigenetic modification level is a histone-type modification.

Embodiment 62. The method of Embodiment 52, wherein the condition is a predicted age of the tissue sample.

Embodiment 63. The method of Embodiment 52, wherein the condition is a level of cellular senescence in the tissue sample.

Embodiment 64. The method of Embodiment 63, wherein the level of cell senescence is indicative of cell apoptosis in the tissue sample.

Embodiment 65. The method of Embodiment 63, wherein the level of cell senescence is indicative of cell necrosis in the tissue sample.

Embodiment 66. The method of Embodiment 52, wherein the condition is a level of oxidative stress in the tissue sample.

Embodiment 67. The method of Embodiment 62, further including: classifying the prospective age-preventing agent as an age-preventing agent if the score determined for the tissue sample in the first vessel is less than the score determined for the tissue sample in the second vessel.

Embodiment 68. The method of Embodiment 52, wherein the tissue age inducing agent and the prospective age-preventing agent are applied simultaneously to the tissue sample in the first vessel.

Embodiment 69. A method for identifying age-reversing agents for biological tissue, comprising: transferring aged tissue samples to a first vessel and a second vessel; applying a prospective age-reversing agent to the aged tissue sample in the first vessel; extracting genetic material from the aged tissue samples in the first vessel and the second vessel; measuring a quantity of a tissue age biomarker in the extracted genetic material from each vessel; and determining a score for the aged tissue samples in the first vessel and the second vessel based on the quantity of the tissue age biomarker measured in the genetic material extracted from the aged tissue sample in each vessel, wherein the score is indicative of a condition of the aged tissue sample in each vessel.

Embodiment 70. The method of Embodiment 69, further including: applying a tissue age inducing agent to a donor tissue sample to create the aged tissue sample.

Embodiment 71. The method of Embodiment 69, further including: isolating aged cells from a donor tissue sample; transferring the isolated aged tissue cells to an incubation vessel; and incubating the aged tissue cells under conditions that promote growth of the aged tissue cells into the aged tissue sample.

Embodiment 72. The method of Embodiment 71, wherein the donor tissue sample is obtained from a donor suffering from AMD.

Embodiment 73. The method of Embodiment 71, wherein the donor tissue sample is obtained from a donor with dementia.

Embodiment 74. The method of Embodiment 69, further including: incubating a donor tissue sample under conditions that promote cell replication to create the aged tissue sample.

Embodiment 75. The method of Embodiment 69, wherein the tissue senescence biomarker is RNA.

Embodiment 76. The method of Embodiment 75, wherein the RNA is messenger RNA (mRNA).

Embodiment 77. The method of Embodiment 75, wherein the RNA is a non-coding RNA.

Embodiment 78. The method of Embodiment 77, wherein the non-coding RNA is siRNA.

Embodiment 79. The method of Embodiment 77, wherein the non-coding RNA is microRNA.

Embodiment 80. The method of Embodiment 69, wherein the tissue age biomaker is an epigenetic modification

Embodiment 81. The method of Embodiment 80, wherein the epigenetic modification is a covalent-type modification.

Embodiment 82. The method of Embodiment 81, wherein the covalent-type modification is methylation.

Embodiment 83. The method of Embodiment 80, wherein the epigenetic modification is a histone-type modification.

Embodiment 84. The method of Embodiment 69, wherein the condition is a predicted age of the aged tissue sample.

Embodiment 85. The method of Embodiment 69, wherein the condition is a level of cellular senescence in the aged tissue sample.

Embodiment 86. The method of Embodiment 85, wherein the level of cell senescence is indicative of cell apoptosis in the aged tissue sample.

Embodiment 87. The method of Embodiment 85, wherein the level of cell senescence is indicative of cell necrosis in the aged tissue sample.

Embodiment 88. The method of Embodiment 69, wherein the condition is a level of oxidative stress in the aged tissue sample.

Embodiment 89. The method of Embodiment 69, further including:

classifying the prospective age-reversing agent as an age-reversing agent if the score determined for the aged tissue sample in the first vessel is less than the score determined for the aged tissue sample in the second vessel.

Embodiment 90. A method for predicting tissue age, comprising: extracting genetic material from a tissue sample; measuring a quantity of a tissue age biomarker in the extracted genetic material; and determining a score for the tissue sample based on the quantity of the tissue age biomarker measured in the extracted genetic material, wherein the score is indicative of a condition of the tissue sample.

Embodiment 91. The method of Embodiment 90, wherein the tissue age biomarker is RNA.

Embodiment 92. The method of Embodiment 91, wherein the RNA is messenger RNA (mRNA).

Embodiment 93. The method of Embodiment 91, wherein the RNA is a non-coding RNA.

Embodiment 94. The method of Embodiment 93, wherein the non-coding RNA is siRNA.

Embodiment 95. The method of Embodiment 93, wherein the non-coding RNA is microRNA.

Embodiment 96. The method of Embodiment 90, wherein the tissue age biomaker is an epigenetic modification

Embodiment97. The method of Embodiment 96, wherein the epigenetic modification is a covalent-type modification.

Embodiment 98. The method of Embodiment 97, wherein the covalent-type modification is methylation.

Embodiment 99. The method of Embodiment 96, wherein the epigenetic modification is a histone-type modification.

Embodiment 100. The method of Embodiment 90, wherein the condition is a predicted age of the tissue sample.

Embodiment 101. The method of Embodiment 93, wherein the non-coding RNA is a long non-coding RNA.

What is claimed:

1. A method for identifying age-preventing agents for skin, comprising:
transferring skin samples to a first vessel, a second vessel, and a third vessel;
applying a skin age inducing agent to the skin samples in the first vessel and the second vessel;
applying a prospective age-preventing agent to the skin sample in the first vessel;
extracting genetic material from the skin samples in the first vessel, the second vessel and the third vessel;
measuring a quantity of a skin age biomarker in the extracted genetic material from each vessel; and
determining a score for the skin samples in the first vessel, the second vessel and the third vessel based on the quantity of skin age biomarker measured in the genetic material extracted from the skin sample in each vessel, wherein the score is indicative of a condition of the skin sample in each vessel.

2. The method of claim 1, wherein the skin age biomarker is RNA.

3. The method of claim 1, wherein the skin comprises dermis.

4. The method of claim 1, wherein the skin comprises epidermis.

5. The method of claim 1, wherein the skin age biomarker is an epigenetic modification.

6. The method of claim 5, wherein the epigenetic modification is a covalent-type modification.

7. The method of claim 6, wherein the covalent-type modification is methylation.

8. The method of claim 5, wherein the epigenetic modification level is a histone-type modification.

9. The method of claim 1, wherein the condition is a predicted age of the skin sample.

10. The method of claim 1, wherein the condition is a level of cellular senescence in the skin sample.

11. The method of claim 1, wherein the skin age biomarker comprises genes or gene expression products comprising mRNA or protein.

12. The method of claim 1, wherein the skin age biomarker includes changes in expression of one or more of the genes in Table 1.
TABLE 1
List of markers associated with aging

| | | | ENTREZ | NCBI | |
|---|---|---|---|---|---|
| | | | Accession | Accession | Correlation |
| Tissue | Marker | Full name | Gene | mRNA | w/aging |
| Skin | P16 | cyclin-dependent kinase | 1029 | NM_000077; | Positive |
| | | inhibitor 2A, multiple tumor | | NM_001195132; | |
| | | suppressor 1 | | NM_058195; | |
| | | | | NM_058196; | |
| | | | | NM_058197 | |
| Skin | IL8 | Interleukin 8 | 3576 | NM_000584; | Positive |
| | | | | NM_001354840 | |
| Skin | MMP-1 | Matrix metalloproteinase-1 | 4312 | NM_002421; | Positive |
| | | | | NM_001145938 | |
| Skin | HAS-2 | Hyaluronan synthase 2 | 3037 | NM_005328 | Negative |
| Skin | ZIC1 | Zinc finger of the | 7545 | NM_003412 | Positive |
| | | cerebellum 1 | | | |
| Skin | BLIMP1 | PR domain zinc finger | 639 | NM_001198; | Positive |
| | | protein 1 | | NM_182907 | |
| Skin | KI67 | Antigen KI-67 | 4288 | NM_001145966; | Negative |
| | | | | NM_002417 | |
| Skin | ZYG11B | Zyg-11 family member B, | 79699 | NM_024646 | Positive |
| | | cell cycle regulator | | | |

13. The method of claim 1, wherein the skin age biomarkers include at least 1, 2, 3, 4, 5, 6, 7 or 8 of the markers of Table 1.

14. The method of claim 1, wherein the skin age biomarkers include at least 1, 2, or 3 of the following markers: ZIC1, BLIMP1 or ZYG11B.

15. The method of claim 1, wherein the skin age biomarkers include 1, 2, or 3 of the following markers: ZIC1, BLIMP1 or ZYG11B and at least 1, 2, 3, 4, or 5 of the following markers: P16; IL8; MMP-1; HAS-2; and/or KI67.

16. The method of claim 1, wherein the skin age biomarkers include signatures, wherein the signature includes signature 1 comprising at least 1, 2, 3, 4, or 5 of the following genes: P16;
IL8; MMP-1; HAS-2; and/or ZIC1; especially, ZIC-1; signature 2 comprising at least 1, 2, 3, 4, or 5 of the following genes: P16; IL8; BLIMP1; KI67 and ZYG11B; especially, BLIMP1 or ZYG11B or both BLIMP1 and ZYG11B; signature 3 comprising at least 1, 2, 3, 4, 5, or 6 positively correlated genes comprising P16; IL8; MMP-1; ZIC1; BLIMP1 and/or ZYG11B or signature 4 comprising at least 1 or 2 negatively correlated genes comprising HAS2 and/or KI67.

17. The method of claim 1, wherein the skin age biomarkers include genes that are weighed differently with respect to the aging parameter being measured.

18. The method of claim 17, wherein the weighted genes comprise, from high to low, P16, ZIC-1, MMP-1, HAS-2, and IL-8.

19. The method of claim 17, wherein the weighted genes comprise, from high to low, BLIMP-1, P16, ZYG11B, IL-8, and KI-67.

20. The method of claim 1, wherein the skin age biomarker includes changes in expression of one or more of the genes in the dermis, as listed in Table 2
TABLE 2
List of markers associated with aging in dermis

| | | | ENTREZ | NCBI | |
|---|---|---|---|---|---|
| | | | Accession | Accession | Correlation |
| Tissue | Marker | Full name | Gene | mRNA | w/aging |
| Dermis | P16 | cyclin-dependent kinase | 1029 | NM_000077; | Positive |
| | | inhibitor 2A, multiple tumor | | NM_001195132; | |
| | | suppressor 1 | | NM_058195; | |
| | | | | NM_058196; | |
| | | | | NM_058197 | |
| Dermis | IL8 | Interleukin 8 | 3576 | NM_000584; | Positive |
| | | | | NM_001354840 | |
| Dermis | MMP-1 | Matrix metalloproteinase-1 | 4312 | NM_002421; | Positive |
| | | | | NM_001145938 | |
| Dermis | HAS-2 | Hyaluronan synthase 2 | 3037 | NM_005328 | Negative |
| Dermis | ZIC1 | Zinc finger of the | 7545 | NM_003412 | Positive |
| | | cerebellum 1 | | | |

21. The method of claim 20, wherein the markers include at least 1, 2, 3, 4, or 5 of the markers of Table 2.

22. The method of claim 20, wherein the marker includes at least ZIC1.

23. The method of claim 20, wherein the marker includes ZIC1 and at least 1, 2, 3, or 4 of the following markers: P16; IL8; MMP-1; and/or HAS-2.

24. The method of claim 20, wherein the marker includes a signature comprising signature 1 comprising at least 1, 2, 3, 4, or 5 of the following genes: P16; IL8; MMP-1; HAS-2; and/or ZIC1; or signature 2 comprising 1, 2, 3, or 4, positively correlated genes comprising P16;
IL8; MMP-1; and/or ZIC1.

25. The method of claim 20, wherein the markers are weighted differently with respect to the aging parameter being measured.

26. The method of claim 25, wherein the markers are weighted, from high to low, as follows:
P16, ZIC-1, MMP-1, HAS-2, and 11-8.

27. The method of claim 1, wherein the skin age biomarker includes changes in expression of one or more of the genes in the epidermis, as listed in Table 3
TABLE 3
List of markers associated with aging in epidermis

| | | | NCBI | NCBI | |
|---|---|---|---|---|---|
| | | | Accession | Accession | Correlation |
| Tissue | Marker | Full name | Gene | mRNA | w/aging |
| Epidermis | P16 | cyclin-dependent kinase | 1029 | NM_000077; | Positive |
| | | inhibitor 2A, multiple tumor | | NM_001195132; | |
| | | suppressor 1 | | NM_058195; | |
| | | | | NM_058196; | |
| | | | | NM_058197 | |
| Epidermis | IL8 | Interleukin 8 | 3576 | NM_000584; | Positive |
| | | | | NM_001354840 | |
| Epidermis | BLIMP1 | PR domain zinc finger | 639 | NM_001198; | Positive |
| | | protein 1 | | NM_182907 | |
| Epidermis | KI67 | Antigen KI-67 | 4288 | NM_001145966; | Negative |
| | | | | NM_002417 | |
| Epidermis | ZYG11B | Zyg-11 family member B, | 79699 | NM_024646 | Positive |
| | | cell cycle regulator | | | |

28. The method of claim 27, wherein the skin age biomarker includes at least 1, 2, 3, 4, or 5 of the markers of Table 3.

29. The method of claim 27, wherein the skin age biomarker includes at least ZYG11B or BLIMP1 or both ZYG11B and BLIMP1.

30. The method of claim 27, wherein the skin age biomarker includes ZYG11B or BLIMP1 or both ZYG11B and BLIMP1 and at least 1, 2, or 3 of the following markers: P16; IL8;
and/or KI67.

31. The method of claim 27, wherein the skin age biomarker includes a signature comprising signature 1 comprising at least 1, 2, 3, 4, or 5 of the following genes: ZYG11B, BLIMP1 P16; IL8; and/or KI67; or signature 2 comprising 1, 2, 3, or 4, positively correlated genes comprising P16; IL8; ZYG11B; and/or BLIMP1.

32. The method of claim 27, wherein the biomarkers are weighted differently with respect to the aging parameter being measured.

33. The method of claim 32, wherein the biomarkers are weighed, from high to low, as follows: BLIMP-1, P16, ZYG11B, IL-8, and KI-67.

34. The method of claim 9, further including:
classifying the prospective age-preventing agent as an age-preventing agent if the score determined for the skin sample in the first vessel is less than the score determined for the skin sample in the second vessel.

35. The method of claim 1, wherein the skin age inducing agent and the prospective age-preventing agent are applied simultaneously to the skin sample in the first vessel.

36. A method for identifying age-reversing agents for skin, comprising:
transferring aged skin samples to a first vessel and a second vessel;
applying a prospective age-reversing agent to the aged skin sample in the first vessel;
extracting genetic material from the aged skin samples in the first vessel and the second vessel;
measuring a quantity of a skin age biomarker in the extracted genetic material from each vessel; and
determining a score for the aged skin samples in the first vessel and the second vessel based on the quantity of the skin age biomarker measured in the genetic material extracted from the aged skin sample in each vessel, wherein the score is indicative of a condition of the aged skin sample in each vessel.

37. The method of claim 36, further including:
applying a skin age inducing agent to a donor skin sample to create the aged skin sample.

38. The method of claim 36, further including:
isolating aged skin cells from a donor skin sample;
transferring the isolated aged skin cells to an incubation vessel; and
incubating the aged skin cells under conditions that promote growth of the aged skin cells into the aged skin sample.

39. The method of claim 36, further including:
incubating a donor skin sample under conditions that promote cell replication to create the aged skin sample.

40. The method of claim 36, wherein the skin senescence biomarker is RNA.

41. The method of claim 36, wherein the condition is a predicted age of the aged skin sample.

42. The method of claim 36, wherein the condition is a level of cellular senescence in the aged skin sample.

43. The method of claim 36, further including:
classifying the prospective age-reversing agent as an age-reversing agent if the score determined for the aged skin sample in the first vessel is less than the score determined for the aged skin sample in the second vessel.

44. A method for predicting skin age, comprising:
extracting genetic material from a skin sample;
measuring a quantity of a skin age biomarker in the extracted genetic material; and
determining a score for the skin sample based on the quantity of the skin age biomarker measured in the extracted genetic material, wherein the score is indicative of a condition of the skin sample.

45. The method of claim 44, wherein the skin age biomarker is RNA.

46. The method of claim 44, wherein the condition is a predicted age of the skin sample.

47. The method of claim 46, wherein the non-coding RNA is a long non-coding RNA.

48. The method of claim 37, wherein the skin age inducing agent is selected from the group consisting of doxorubicin, hydrogen peroxide and ionizing radiation.

49. A method for identifying age-preventing agents for tissue, comprising:
transferring tissue samples to a first vessel, a second vessel, and a third vessel;
applying a tissue age inducing agent to the tissue samples in the first vessel and the second vessel;
applying a prospective age-preventing agent to the tissue sample in the first vessel;
extracting genetic material from the tissue samples in the first vessel, the second vessel and the third vessel;
measuring a quantity of a tissue age biomarker in the extracted genetic material from each vessel; and
determining a score for the tissue samples in the first vessel, the second vessel and the third vessel based on the quantity of tissue age biomarker measured in the genetic material extracted from the tissue sample in each vessel, wherein the score is indicative of a condition of the tissue sample in each vessel.

50. The method of claim 49, wherein the tissue age biomarker is RNA.

51. The method of claim 49, wherein the condition is a predicted age of the tissue sample.

52. The method of claim 49, wherein the condition is a level of cellular senescence in the tissue sample.

53. The method of claim 52, wherein the level of cell senescence is indicative of senescence associated beta-galactosidase positive cells in the tissue sample.

54. The method of claim 52, wherein the level of cell senescence is indicative of p16 positive cells in the tissue sample.

55. The method of claim 49, wherein the condition is a level of oxidative stress in the tissue sample.

56. The method of claim 49, further including:
classifying the prospective age-preventing agent as an age-preventing agent if the score determined for the tissue sample in the first vessel is less than the score determined for the tissue sample in the second vessel.

57. The method of claim 49, wherein the tissue age inducing agent and the prospective age-preventing agent are applied simultaneously to the tissue sample in the first vessel.

58. A method for identifying age-reversing agents for biological tissue, comprising:
transferring aged tissue samples to a first vessel and a second vessel;
applying a prospective age-reversing agent to the aged tissue sample in the first vessel;
extracting genetic material from the aged tissue samples in the first vessel and the second vessel;
measuring a quantity of a tissue age biomarker in the extracted genetic material from each vessel; and
determining a score for the aged tissue samples in the first vessel and the second vessel based on the quantity of the tissue age biomarker measured in the genetic material extracted from the aged tissue sample in each vessel, wherein the score is indicative of a condition of the aged tissue sample in each vessel.

59. The method of claim 58, further including:
applying a tissue age inducing agent to a donor tissue sample to create the aged tissue sample.

60. The method of claim 58, further including:
isolating aged cells from a donor tissue sample;
transferring the isolated aged tissue cells to an incubation vessel; and
incubating the aged tissue cells under conditions that promote growth of the aged tissue cells into the aged tissue sample.

61. The method of claim 60, further including:
incubating a donor tissue sample under conditions that promote cell replication to create the aged tissue sample.
